(19) United States
(12) Patent Application Publication
(10) Pub. No.: US 2007/0254381 A1
(43) Pub. Date: Nov. 1, 2007

(54) Monitoring and/or treating syringe mechanism

(75) Inventors: Edward K.Y. Jung, Bellevue, WA (US); Eric C. Leuthardt, St Louis, MO (US); Royce A. Levien, Lexington, MA (US); Robert W. Lord, Seattle, WA (US); Mark A. Malamud, Seattle, WA (US); John D. Rinaldo JR., Bellevue, WA (US); Lowell L. Wood JR., Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/413,973

(22) Filed: Apr. 27, 2006

(51) Int. Cl. G01N 33/558

(52) U.S. Cl. 436/514; 514/2

(57) ABSTRACT

One aspect relates to analyzing at least one condition of at least one fluid as contained within an individual, and transferring at least one element relative to the individual in an effort to maintain and/or change the at least one condition of the at least one element with respect to the at least one fluid at least partially in response to the analyzing the at least one condition of the at least one fluid as contained within the individual. Another aspect relates to monitoring at least one fluid(s) and/or element(s) of an individual; and adaptively treating the individual at least partially by determining whether to modify the at least one fluid(s) and/or element(s) within the individual at least partially in response to the monitoring the at least one fluid(s) and/or element(s) of the individual.

### TECHINICAL FIELD

Certain aspects of this disclosure can relate to, but are not limited to, a mechanism or technique that can monitor at least one fluid(s) and/or element(s), or at least one element with respect to the element, of an individual (e.g., a human, animal, or organism). Certain aspects of this disclosure can relate to, but are not limited to, a mechanism or technique that can treat the individual.

### BRIEF DESCRIPTION OF THE FIGURES

FIG. 1 is a block diagram of one embodiment of a monitoring and/or treating syringe mechanism;

FIG. 2 is a diagram of another embodiment of the monitoring and/or treating syringe mechanism;

FIG. 3 is a diagram of another embodiment of the monitoring and/or treating syringe mechanism;

FIG. 4 is a diagram of another embodiment of the monitoring and/or treating syringe mechanism;

FIG. 5 is a diagram of another embodiment of the monitoring and/or treating syringe mechanism;

FIG. 6 (including FIGS. 6a, 6b, 6c, and 6d) is a flowchart of one embodiment of a monitoring and/or treating technique;

FIG. 7 is a flowchart of another embodiment of a monitoring and/or treating technique;

FIG. 8 illustrates a cross-sectional view of one embodiment of the injecting-probe being configured as a uniaxial needle;

FIG. 9 illustrates a cross-sectional view of another embodiment of the injecting-probe being configured as a coaxial needle;

FIG. 10 illustrates a cross-sectional view of another embodiment of the injecting-probe being configured as a parallel-axis needle;

FIG. 11 illustrates a cross-sectional view of another embodiment of the monitoring and/or treating syringe mechanism including the injecting-probe being configured as a needleless delivery system; and

FIG. 12 is a diagram of another embodiment of the monitoring and/or treating syringe mechanism.

### DETAILED DESCRIIPTION

At least certain portions of the text of this disclosure (e.g., claims and/or detailed description and/or drawings as set forth herein) can support various different applications. Although, for sake of convenience and understanding, the detailed description includes section headings that generally track the titles of the various different supported applications or follow the general concepts contained therein, and is not intended to limit the scope of the invention as set forth by each particular claim. It is to be understood that support for the various applications thereby can appear throughout the text and/or drawings, irrespective of the section headings.

### 1. CERTAIN EMBODIMENTS OF A MONITORING AND/OR TREATING SYRINGE MECHANISM

Certain aspects of this disclosure can relate to a variety of monitoring and/or treating syringe mechanism 100, as well as a variety of techniques associated therewith. Certain embodiments of the monitoring and/or treating syringe mechanism 100 can be configured to analyze and/or monitor at least on fluid(s) and/or element(s) that are contained with or associated with an individual 120. Certain embodiments of the monitoring and/or treating syringe mechanism 100 can be configured to treat and/or disperse at least partially using at least one fluid(s) and/or element(s), relative to the individual 120.

Within this disclosure, the term “at least one fluid(s) and/or element(s)” is not intended be constrained according to typical antecedent basis. For instance, at least one fluid(s) and/or element(s) can be analyzed as withdrawn from or within the individual. Based at least in part upon the analysis of the at least one fluid(s) and/or element(s), the at least one fluid(s) and/or element(s) can be injected into or applied to the individual. The at least one fluid(s) and/or element(s) that have been injected into or applied to the individual may be identical, modified, are completely different from the at least one fluid(s) and/or element(s) as analyzed as withdrawn from or within the individual. Additionally, the at least one fluid(s) and/or element(s) as analyzed as withdrawn from or within the individual can include at least one fluid(s) with no element, at least one element(s) with no fluid, or any combination of fluid(s) and/or element(s). Alternately, the at least one fluid(s) and/or element(s) as injected into or applied to the individual can include at least one fluid(s) with no element, at least one element(s) with no fluid, or any combination of fluid(s) and/or element(s).

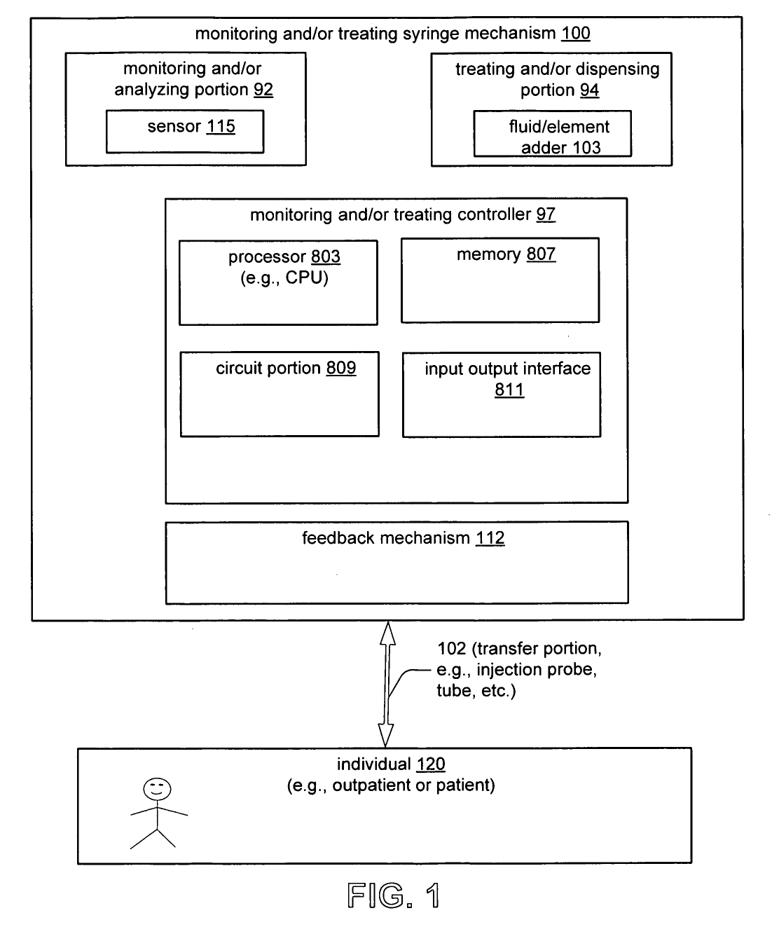

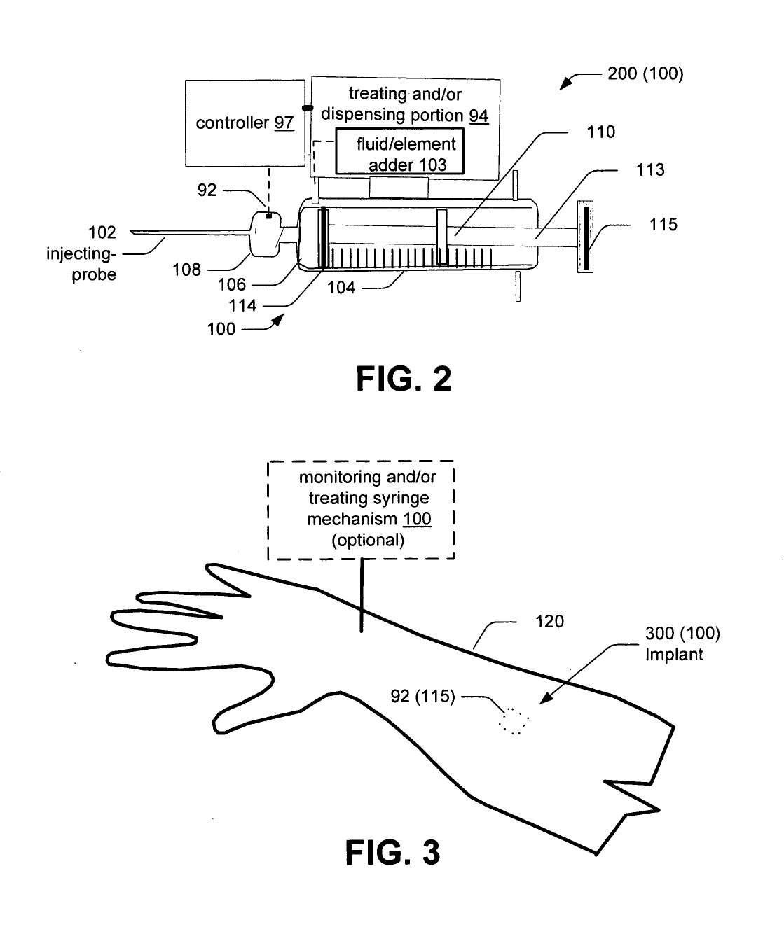

FIG. 1 shows a block diagram of one embodiment of the monitoring and/or treating syringe mechanism 100 that can be used to perform one or more varied operations with respect to at least one fluid(s) and/or element(s) that can include, but is not limited to one or more of: withdrawing or aspirating the at least one fluid(s) and/or element(s) from the individual 120 and/or another source; analyzing or monitoring the at least one fluid(s) and/or element(s); and/or treating the individual at least partially in response to the analyzing. There are a variety of embodiments of the monitoring and/or treating syringe mechanism 100 as described with respect to FIGS. 2 to 5, which may be provided with distinguishing reference characters in addition to 100.

In certain embodiments, the treating can include, but is not limited to, injecting the at least one treatment fluid(s) and/or element(s) into the individual 120 and/or establishing a regimen (medical or other) for the individual. In certain embodiments, a feedback mechanism 112 can be included in the monitoring and/or treating syringe mechanism 100, whereby the treating as can be performed by certain embodiments of the treating and/or dispensing portion 94, and can be altered and/or controlled at least partially in respect to the monitoring and/or analyzing as can be performed by the treating and/or dispensing portion 94. FIGS. 2, 3, 4, and 5 illustrate a number of the embodiments of the monitoring and/or treating syringe mechanism 100 as described with respect to FIG. 1.

Within this disclosure, the term “fluid”, as used herein, may refer to liquids, gases, and other compositions, mixtures, or materials exhibiting fluid behavior. Within this disclosure, the term “fluid” can include, but is not limited to, the fluid that can at least partially be obtained from, at least partially injected into, and/or applied to the individual 120. Certain embodiments of each of the at least one fluid(s) and/or element(s) (and/or a mixture thereof), can include but is not limited to: one or more liquid(s), one or more solid(s), one or more gases, and/or a combination thereof. Certain embodiments of the at least one fluid(s) and/or element(s) can be utilized for a variety of purposes relative to the individual that can include but are not limited to: associated with the metabolism of the individual, contained within the individual or an organ of the individual, maintaining the life of the individual, being a waste-product of the individual, or being a combination thereof.

Certain embodiments of the fluid within the body of the individual may include a liquid, a gas, a mixture including a liquid, or a mixture including a gas. As used herein, the term fluid may encompass liquids, gases, or mixtures thereof that also include solid particles in a fluid carrier. Certain embodiments of the liquids may include, but are not limited to, mixtures of two or more different liquids, solutions, slurries, or suspensions. Examples of liquids present within the body can include blood, lymph, serum, urine, semen, digestive fluids, tears, ambiotic fluid, amniotic fluid, saliva, mucous, cerebro-spinal fluid, intestinal contents, bile, epithelial exudate, or esophageal contents. Certain embodiments of the liquids can present within the body may include synthetic or introduced liquids, such as blood substitutes or drug, nutrient, or buffered saline solutions. Certain embodiments of the fluids may include liquids containing dissolved gases or gas bubbles, or gases containing fine liquid droplets or solid particles. Certain embodiments of the gases or gaseous mixtures found within the body may include inhaled and exhaled air, e.g. in the nasal or respiratory tract, or intestinal gases.

Within this disclosure, certain embodiments of the term “fluid” can include but is not limited to a “body fluid”. Body fluid components may include, but are not limited to, cells, cellular fractions or components, collections or aggregations of cells, bacterial, viral or fungal species, ions, molecules, gas bubbles, dissolved gas, suspended particles, or a variety of other materials that may be present in the body fluid, are also indicated. Body fluid components may be materials that are normally present in the body fluid, materials that are naturally derived but not normally present in the body fluid, or foreign materials that have entered or been introduced to the body fluid (including but not limited to pathogens, toxins, pollutants, or medications, for example).

Within this disclosure, the term “individual” can include and/or apply to a human, animal, or organism. As is traditional with numerous medical technologies, certain designs, uses, mechanisms, and/or configurations of the monitoring and/or treating syringe mechanism 100 that is intended or designed to be applied to humans can, for example, also be applied to animals or organisms (or vice versa).

Within this disclosure, the term “element” can include, but is not limited to, that which can be mixed with, combined with, integrated in, or otherwise associated with the fluid. For example, gasses, solids, liquids, chemicals, materials, and/or medicines, etc. that can be mixed, combined, suspended, or otherwise associated with the fluid may be considered individually as an embodiment of the element. Additionally, certain naturally-occurring chemicals (e.g., carbon dioxide, oxygen, etc.), elements, viruses, germs, etc. at least partially contained within the fluid may be considered as an individual embodiment of the element. As such, certain embodiments of the at least one element can include a gas, a solid, or a liquid that can be included in, or carried by, the at least one fluid(s) and/or element(s). As such, within this disclosure, the terms “fluid” and “element” should be construed broadly, such as to indicate any fluid and/or element as associated with the individual which can be analyzed, monitored, and/or treated.

Within this disclosure, the term “at least one fluid(s) and/or the at least one element(s)” can include, but is not limited to, one or more fluids, one or more elements, or any combination thereof. Within this disclosure, the term “at least one sensed fluid(s) and/or the at least one sense to element(s)” can include, but is not limited to, one or more fluids, one or more elements, or any combination thereof such as can be sensed by at least a portion of the monitoring and/or treating syringe mechanism 100. Within this disclosure, the term “at least one modified fluid(s) and/or the at least one modified element(s)” can include, but is not limited to, one or more fluids, one or more elements, or any combination thereof that of the modified at least in part based upon any sensed condition of the individual.

By monitoring the at least one fluid(s) and/or the at least one element(s), certain aspects of the individual's condition, health, medication, sickness, etc. can be determined. As such, it is to be understood that the terms “fluid” is to be interpreted extremely broadly, and can be considered as applying even to fluids that could be applied utilizing the relatively broad limits of the phlebotomy, such as can include but are not limited to an injecting-probe 102 (including, but not limited to, a uniaxial needle, a coaxial needle, a parallel-axis needle, a limited-invasion gun injection device), a tube, a patch, and/or other technologies such as can transfer the at least one fluid(s) and/or the at least one element(s), as described in this disclosure.

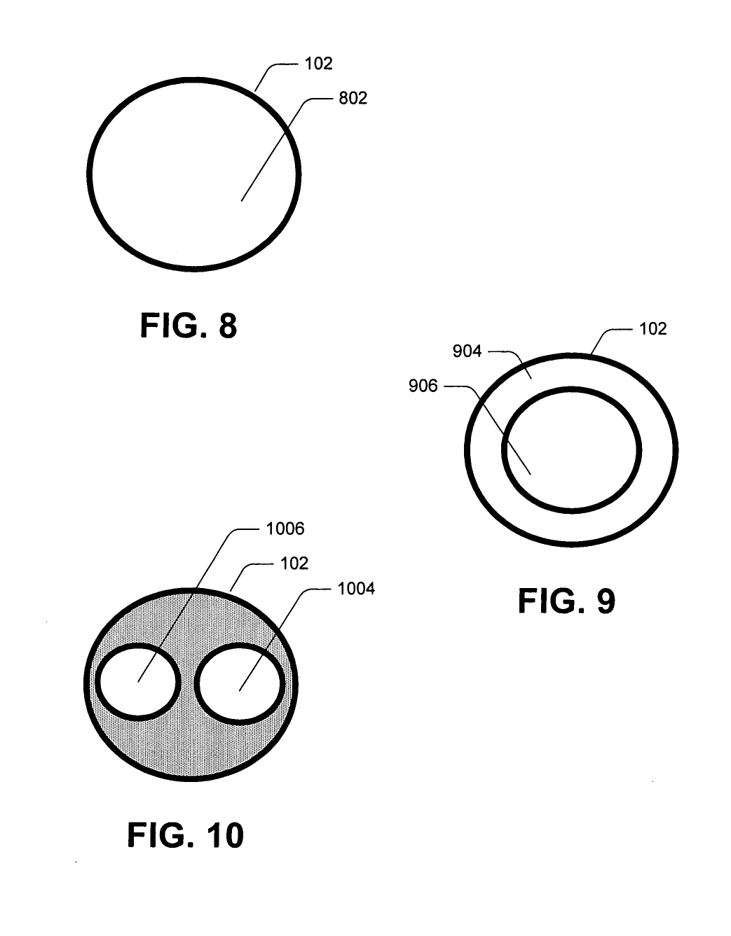

A variety of the injecting-probe embodiments are illustrated with respect to FIGS. 8 to 12, for example. These embodiments of injecting-probes are intended to be illustrative in nature and not limiting in scope. FIG. 8 illustrates a cross-sectional view of one embodiment of the injecting-probe 102 being configured as a uniaxial needle which includes a single lumen 802 extending the length of the needle. With certain embodiments of the monitoring and/or treating syringe mechanism 100 including the uniaxial needle, the at least one fluid(s) and/or element(s) can travel within and along both axial directions of the uniaxial needle within the lumen 802, depending on the operation of the monitoring and/or analyzing portion 92 and/or the treating and/or dispensing portion 94 in respectively withdrawing and/or injecting the at least one fluid(s) and/or element(s).

FIG. 9 illustrates a cross-sectional view of another embodiment of the injecting-probe being configured as a coaxial needle which in certain embodiments can include a first lumen 904 and a second lumen 906, which both extend substantially the length of the coaxial needle. In certain embodiments, the first lumen 904 can be in fluid communication with the monitoring and/or analyzing portion 92 to allow the at least one fluid(s) and/or element(s) to be withdrawn from the individual. In certain embodiments, the second lumen 906 can be in fluid communication with the treating and/or dispensing portion 94 to allow the at least one fluid(s) and/or element(s) to be injected into the individual. In certain embodiments, first lumen 904 can be fluid communication with the treating and/or dispensing portion 94, while the second lumen 906 can be in fluid communication with the monitoring and/or analyzing portion 92.

FIG. 10 illustrates a cross-sectional view of another embodiment of the injecting-probe 102 being configured as a parallel-axis needle which can include a first lumen 1004 and a second lumen 1006, which both extend substantially the length of the coaxial needle. In certain embodiments, the first lumen 1004 can be in fluid communication with the monitoring and/or analyzing portion 92 to allow the at least one fluid(s) and/or element(s) to be withdrawn from the individual. In certain embodiments, the second lumen 1006 can be in fluid communication with the treating and/or dispensing portion 94 to allow the at least one fluid(s) and/or element(s) to be injected into the individual. In certain embodiments, the first lumen 1004 can be in fluid communication with the treating and/or dispensing portion 94, while the second lumen 1006 can be in fluid communication with the monitoring and/or analyzing portion 92. Certain embodiments of the injecting probe 102 can be configured with two or more lumens or channels, but only certain ones of which are used during operation of the monitoring and/or treating syringe mechanism 100. Perhaps certain ones of the lumens or channels can be used in an alternate configuration, or may not be used at a particular time. For instance, based on analysis, it may be determined that the condition of the at least one fluid(s) and/or element(s) is currently acceptable, and does not need to be modified.

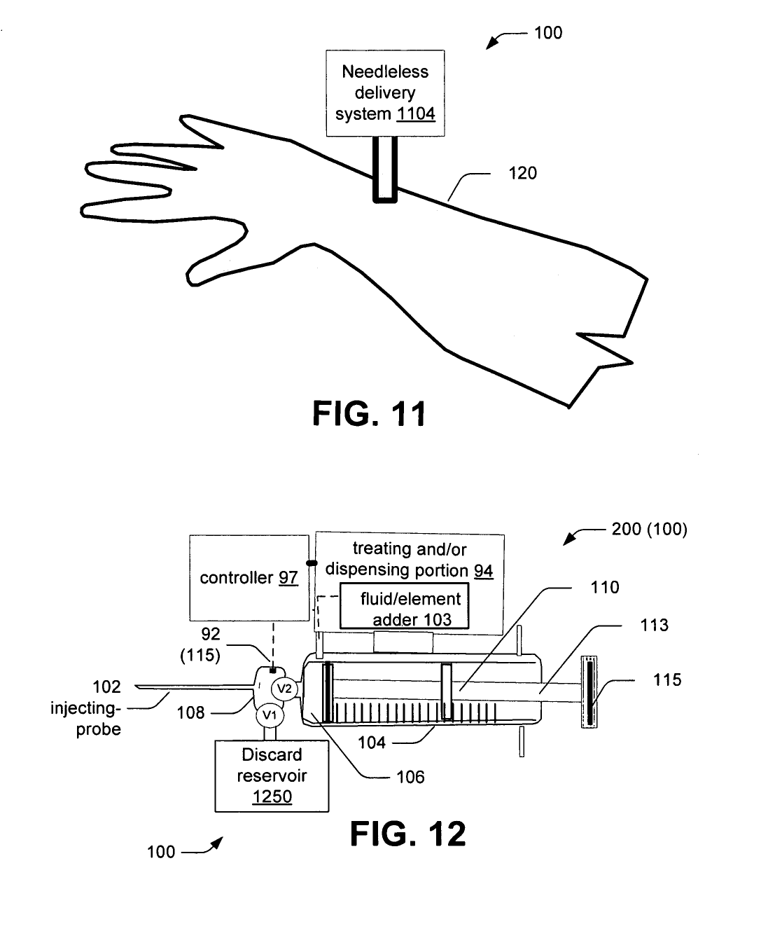

FIG. 11 illustrates a cross-sectional view of another embodiment of the injecting-probe 102 being configured as a needleless delivery system 1104 which can inject the at least one fluid and/or element into the individual (e.g., subcutaneous or intradermal tissue). There are a variety of embodiments of needleless delivery systems such as described with respect to U.S. Pat. No. 6,893,657; U.S. Pat. No. 6,811,792; U.S. Pat. No. 6,673,034; U.S. Pat. No. 6,565,871; U.S. Pat. No. 6,063,053; U.S. Pat. No. 6,056,716 that can be used to inject at least one fluid and/or element, and such needleless delivery systems are intended to be within the intended scope of the present disclosure as set forth in the claims. With certain embodiments of the needleless delivery systems, the at least one fluid(s) and/or element(s) can be injected as a liquid, a fluid, a gas, a capsule, a solid dose, etc.

Certain embodiments of the monitoring and/or treating syringe mechanism 100 can be utilized or operated by the individual and/or a phlebotomist. Within this disclosure, the term “phlebotomist” or “phlebotomy” may be intended, depending upon context, to be applied to an individual (e.g., a patient or person utilizing the monitoring in/for treating mechanism) for whom the at least one fluid(s) and/or element(s) is withdrawn and/or injected at least partially utilizing at least one injecting-probe, at least one patch, at least one implant, and/or another suitable monitoring and/or treating syringe mechanism 100. The term “phlebotomist” or “phlebotomy” can also apply to the person who inject, applies, monitors, treats, and/or withdraws the at least one fluid(s) and/or element(s) into/from the individual using at least one embodiment of the monitoring and/or treating syringe mechanism 100. As such, certain embodiments of phlebotomy can include, but is not limited to: a patient, a doctor, a nurse, an ambulance attendee, an emergency health-care provider, the individual, a friend, a family member, or an associate of the individual, etc. As such, phlebotomy as described within the disclosure applies more to a practicing operator of the monitoring and/or treating syringe mechanism 100 as compared to (though not intended to exclude) a licensed doctor, nurse, phlebotomist, etc.

Certain embodiments of the monitoring and/or treating syringe mechanism 100, as described with respect to FIG. 1, can include, but is not limited to: a monitoring and/or analyzing portion 92, a treating and/or dispensing portion 94, a monitoring and/or treating controller 97, and/or a feedback mechanism 112. Certain embodiments of the monitoring and/or analyzing portion 92 can include a sensor 115 that can be configured to analyze the condition of the at least one fluid(s) and/or element(s) that can be withdrawn from, aspirated from, injected into, applied to, or remain within the individual. Certain embodiments of the analyzing the at least one fluid(s) and/or element(s) can be to monitor and/or one or more particular conditions of the individual. Within this disclosure, the terms “condition” relating to the individual can include, but are not limited to: an illness, a sickness, an organism, a cancer, an immune deficiency, etc., or other such conditions that can be monitored, analyzed, and/or treated at least partially using certain embodiments of the monitoring and/or treating syringe mechanism 100.

Certain embodiments of the sensor 115 can include, but is not limited to: an optical sensor, an imaging device, an acoustic sensor, a pressure sensor, a temperature sensor, a flow sensor, a viscosity sensor, or a shear sensor for measuring the effective shear modulus of the fluid at a frequency or strain-rate, a chemical sensor for determining the concentration of a chemical compound or species, a biosensor, and an electrical sensor, for example. Certain embodiments of an optical sensor may be configured to measure the optical absorption, optical emission, fluorescence, electromagnetic radiated commissions, or phosphorescence of at least a portion of the fluid of the fluid, for example. Such optical properties may be inherent optical properties of all or a portion of the fluid, or may be optical properties of materials added or introduced to the fluid, such as tags or markers for materials of interest within the fluid. Certain embodiments of a biosensor may detect materials including, but not limited to, a biological marker, an antibody, an antigen, a peptide, a polypeptide, a protein, a complex, a nucleic acid, a cell (and, in some cases, a cell of a particular type, e.g. by methods used in flow cytometry), a cellular component, an organelle, a gamete, a pathogen, a lipid, a lipoprotein, an alcohol, an acid, an ion, an immunomodulator, a sterol, a carbohydrate, a polysaccharide, a glycoprotein, a metal, an electrolyte, a metabolite, an organic compound, an organophosphate, a drug, a therapeutic, a gas, a pollutant, or a tag. Certain embodiments of a biosensor may include an antibody or other binding molecule such as a receptor or ligand. Sensor 115 may include a single sensor or an array of sensors, and is not limited to a particular number or type of sensors. Certain embodiments of the sensor 115 might comprise in part or whole, a gas sensor such as an acoustic wave, chemiresistant, or piezoelectric sensor, a liquid sensor, or perhaps an electronic nose. Certain embodiments of the sensor 115 may be very small, comprising a sensor or array that is a chemical sensor (Chemical Detection with a Single-Walled Carbon Nanotube Capacitor E. S. Snow, 2005 Science Vol. 307; 1942-1945), a gas sensor (Smart single-chip gas sensor microsystem Hagleitner, C. et al. 2001 NATURE VOL 414 p. 293-296.), an electronic nose, a nuclear magnetic resonance imager (“Controlled multiple quantum coherences of nuclear spins in a nanometre-scale device”, Go Yusa, 2005, Nature 343: 1001-1005). Further examples of sensors that can be utilized as the sensor 115 are provided in The Biomedical Engineering Handbook, Second Edition, Volume I, J. D. Bronzino, Ed., Copyright 2000, CRC Press LLC, pp. V-1-51-9, and U.S. Pat. No. 6,802,811, both of which are incorporated herein by reference. Certain embodiments of the sensor 115 may be configured to measure various parameters, including, but not limited to, the electrical resistivity of the fluid, the density or sound speed of the fluid, the pH, the osmolality, or the index of refraction of the fluid of at least one wavelength. The selection of a suitable sensor for a particular application or use site is considered to be within the capability of a person having skill in the art.

Certain embodiments of the structural elements depicted in FIGS. 2-5 are intended to serve as examples, and are in no way limiting. The choice of structural element size and configuration appropriate for the particular body of the individual may be selected by a person of skill in the art. Structural elements may be constructed by a variety of manufacturing methods, from a variety of materials. Appropriate materials may include metals, ceramics, polymers, and composite materials having suitable biocompatibility, sterilizability, mechanical, and physical properties, as will be known to those of skill in the art. Examples of materials and selection criteria are described, for example, in The Biomedical Engineering Handbook, Second Edition, Volume I, J. D. Bronzino, Ed., Copyright 2000, CRC Press LLC, pp. IV-1-43-31. Manufacturing techniques may include injection molding, extrusion, die-cutting, rapid-prototyping, etc., and will depend on the choice of material and device size and configuration. Sensing and active portions of the lumenally-active device as well as associated electrical circuitry (not depicted in FIGS. 2-5) may be fabricated on the structural element using various microfabrication and/or MEMS techniques, or may be constructed separately and subsequently assembled to the structural element, as one or more distinct components.

Certain embodiments of the treating and/or dispensing portion 94 can be configured to provide treatment for a detected illness, sickness, etc. such as by dispensing the at least one fluid(s) and/or element(s). Such monitoring and/or analysis can be performed on a variety of the at least one fluid(s) and/or element(s) relating to each particular illness, sickness, etc. based, for example, on the treatment and/or regimen. Certain embodiments of the monitoring and/or treating syringe mechanism 100, as described with respect to FIG. 1, can include a feedback mechanism 112 that can monitor a condition of the individual and/or the at least one fluid and/or element of the individual, and provide a suitable treatment or regimen of treatment at least partially in response to the monitored condition. Certain embodiments of the feedback mechanism can thereby be utilized at a location or region remote of a physician, hospital, clinic, etc.; and can thereby be considered to provide an adoptive medical treatment or regimen.

Certain embodiments of the treating and/or dispensing portion 94 can be configured to, at least partially based upon the analysis, inject or provide with the at least one fluid(s) and/or element(s) into the individual. At least certain portions or certain embodiments of the treating and/or dispensing portion 94 can be configured as a syringe as described in this disclosure with respect to FIGS. 2 and 5.

A variety of embodiments of the monitoring and/or treating syringe mechanism 100 can be configured or utilized to perform a variety of the following operations, only certain ones of the operations, each of these operations plus additional functions, or only certain ones of these operations and additional functions. Certain embodiments of the monitoring and/or treating syringe mechanism 100 can at least be partially configured to analyze the at least one fluid(s) and/or element(s) that can be withdrawn from the individual, and/or at least partially based upon the analysis, inject the at least one fluid(s) and/or element(s) into the individual. Certain other embodiments of the monitoring and/or treating syringe mechanism 100 can perform only certain ones of these operations, and may interact with other embodiments of the monitoring and/or treating syringe mechanism that can, for example, perform certain prescribed operations as described in this disclosure. Certain embodiments of the monitoring and/or treating syringe mechanism 100 can be configured to analyze the at least one fluid(s) and/or element(s) within the individual, and at least partially based upon the analysis, provide at least one modified fluid(s) and/or element(s) within the individual. As such, certain embodiments of the monitoring and/or treating syringe mechanism 100 can perform a variety of, or all of the operations, associated with the monitoring and/or treating syringe mechanism 100.

Certain embodiments of the monitoring and/or treating syringe mechanism 100 can perform only certain ones of the operations, and as such can interface with other embodiments and/or portions of the monitoring and/or treating application. For example, certain embodiments of the monitoring and/or treating syringe mechanism 100 such as is at least partially internal to the individual 120, as illustrated with respect to FIG. 2 can monitor and/or analyze the at least one fluid(s) and/or element(s) within the individual, and transfer the results of the monitoring and/or analysis to another portion of the monitoring and/or treating syringe mechanism 100 (or alternately to another monitoring and/or treating syringe mechanism 100 that may be at least partially situated outside of the individual 120). As such, the results of the monitoring and/or analysis can be transferred within a variety of different embodiments of the monitoring and/or transferring mechanism; or alternately different portions of the monitoring and/or treating syringe mechanism 100 as described with respect to FIGS. 1, 2, 3, 4, and/or 5. Thereupon, certain embodiments of the monitoring and/or treating syringe mechanism 100 can be configured to determine the at least one fluid(s) and/or element(s) that should be added to, applied to, or injected into the individual for treatment, and thereupon inject, apply and/or add the at least one modified fluid(s) and/or element(s) into the individual based at least in part on the monitoring and/or analysis.

Similarly, certain embodiments of the monitoring and/or treating syringe mechanism 100 can be configured to analyze the at least one fluid(s) and/or element(s) that can be maintained and/or changed within the individual (e.g., without providing any of the at least one fluid(s) and/or element(s) within the individual). In certain embodiments, another monitoring and/or treating syringe mechanism can utilize the information, instructions, etc. from another one of the monitoring and/or treating syringe mechanism, and thereupon add the at least one modified fluid(s) and/or element(s) into the individual based at least in part on the analysis. As such, certain embodiments of the monitoring and/or treating syringe mechanism 100 can be segmented into “modular” portions or units, each of which are configurable or able to perform only certain of the aspects of the entire monitoring and/or treating syringe mechanism.

Certain operations or tasks performed by certain embodiments of the monitoring and/or treating syringe mechanism 100 may not be performed by other embodiments of the monitoring and/or treating syringe mechanism, or alternately may be performed by a user of the monitoring and/or treating syringe mechanism. For example, based upon input (analysis of the at least one fluid(s) and/or element(s) which may be provided using a variety of user interfaces or information displays) by certain embodiments of the monitoring and/or treating syringe mechanism 100, a user such as a phlebotomist or the individual may decide to inject the at least one modified fluid(s) and/or element(s) into the individual.

Therefore, at least certain ones of the operation(s) that can be performed by the monitoring and/or analyzing portion 92, as well as at least certain ones of the operation(s) that can be performed by the treating and/or dispensing portion 94, can be performed entirely by certain embodiments of the monitoring and/or treating syringe mechanism 100. Alternatively, at least certain ones of the operation(s) that can be performed by the monitoring and/or analyzing portion 92 as well as at least certain ones of the operation(s) that can be performed by the treating and/or dispensing portion 94 can be performed by multiple such modular units that can include but is not limited to certain embodiments of the monitoring and/or treating syringe 200.

FIG. 1 shows one embodiment of the monitoring and/or treating syringe mechanism 100 that can include, among other portions, the monitoring and/or analyzing portion 92 and/or the treating and/or dispensing portion 94. Certain embodiments of the monitoring and/or analyzing portion 92, and a variety of techniques associated therewith as described with respect to this disclosure, can be configured to monitor and/or analyze the at least one fluid(s) and/or element(s) associated with and/or contained within the individual 120. In certain instances, the at least one individual whose at least one fluid(s) and/or at least one element(s) are being analyzed can be a human, or alternately can be an animal or organism.

The embodiment of the monitoring and/or analyzing portion 92 that can be used as described with respect to FIG. 1 can perform a variety of functions with respect to the at least one fluid(s) and/or at least one element(s), which can include but is not limited to: withdrawing or aspirating the at least one fluid(s) and/or at least one element(s) from the individual 120; obtaining the at least one fluid(s) and/or element(s), analyzing the at least one fluid(s) and/or element(s), monitoring the at least one fluid(s) and/or element(s) and/or modifying or generating the at least one fluid(s) and/or element(s). Certain embodiments of the obtaining the at least one fluid(s) and/or element(s) can be based at least in part on the analysis, which can be performed by the monitoring and/or analyzing portion 92, while other embodiments can be performed by the treating and/or dispensing portion 94. For example, certain embodiments of the monitoring and/or analyzing portion 92 can be configured to withdraw, aspirate, and/or analyze the at least one fluid(s) and/or element(s), and based upon the analysis, modify the at least one fluid(s) and/or element(s) to derive the at least one modified fluid(s) and/or element(s), etc. As such, a particular embodiment of the monitoring and/or treating syringe mechanism 100 can be configured or utilized to perform each of these functions; only certain ones of these functions; each of these functions plus additional functions; or only certain ones of these functions plus additional functions.

In certain embodiments, the at least one fluid(s) and/or element(s) that can be withdrawn from, aspirated from, or injected into, the individual may be utilized to attempt to maintain the life of the individual. Such at least one life-maintaining fluid(s) and/or element(s), when withdrawn from, aspirated from, or injected into the individual, should be withdrawn, aspirated, or injected in a careful fashion as to not harm the individual. For example, only certain limited volumes of blood or air should be removed from or applied to the individual. Additionally, care should be applied to providing such life-maintaining fluids as blood, such as by considering blood types, etc. Certain embodiments of the monitoring and/or treating syringe mechanism 100 can thereby monitor the treatment of certain types of individuals such as to ensure that the treatment is being properly provided.

Certain embodiments of the monitoring and/or treating syringe mechanism 100 can be utilized to assist in or improve accepted phlebotomy techniques. For example, certain embodiments of the monitoring and/or treating syringe mechanism 100 can be used to assist in checking, and in certain instances limiting, these instances where inconsistent or incorrect medical treatment may be attempted to be provided. For example, certain embodiments of the monitoring and/or treating syringe mechanism as described in this disclosure could analyze the withdrawn or aspirated blood type, and based on knowledge of the blood to be transfused, ensure that inconsistent blood types and/or components are not combined with the withdrawn blood or components and/or injected back into the individual. A variety of allergies, reactions, illnesses, etc. of each individual could be similarly checked using the monitoring and/or treating syringe mechanism. Also, if a particular monitoring and/or treating syringe mechanism is being configured for a particular individual, the monitoring and/or treating syringe mechanism could be configured to ensure it is being applied to that type of individual.

Certain embodiments of the monitoring and/or treating syringe mechanism 100 may be configured, depending upon context, to assist the individual in maintaining and/or changing a medical regimen such as might have been established by the physician or other medical personnel. Consider that after a particular physicians may have prescribed a particular treatment for certain ones of their individuals and/or patients, then in many instances the individuals and/or patients may fail to follow up on their treatment and/or regimen. The reasons why the individuals and/or patients may fail to follow up on treatments or regimens may include, but is not limited to: complexity of treatment, expense of treatment, requirement to travel to remote locations for treatment (e.g., a doctor's office, clinic, hospital, etc.), the individual's time spent associated within the doctor's office, clinic, hospital, etc. in receiving the treatment, failure of the individual in understanding how to maintain and/or change the treatment or regimen, etc. As such, it may be difficult for the individual to remain on the medical treatment and/or regimen for a variety of reasons.

Certain embodiments of the monitoring and/or treating syringe mechanism 100 can be configured to make it easier to maintain and/or change their medical regimen, treatment, etc. for a particular individual. One important aspect of maintaining and/or changing the medical regimen, treatment, etc. is to monitor the individual's response to the medical regimen, treatment, etc. As such, certain embodiments of the monitoring and/or treating syringe mechanism can be configured to monitor a condition or condition of the individual at least in part by analyzing the at least one fluid(s) and/or element(s) obtained from the individual as described in this disclosure; and/or analyzing elements of the and/or included in the at least one fluid(s) and/or element(s) as described in this disclosure. Certain embodiments of the monitoring and/or treating syringe mechanism therefore allow an individual (and/or an associated phlebotomist) to monitor the at least one fluid(s) and/or element(s) in a manner that in many prior instances could only be performed in a medical office, hospital, etc.

Certain embodiments of the monitoring and/or treating syringe mechanism can include some type of a monitoring communication interface, by which the monitored or analyzed condition of the individual, the at least one fluid(s) included within the individual, and/or one or more element(s) included within the at least one fluid(s) and/or element(s) included within the individual can be communicated to a remote location such as where physicians or other health-care individuals can be situated. Thereupon, in certain embodiments, a physician or other health-care individual can respond by, for example, analyzing the monitored condition of the individual, their fluid(s), and/or their element(s); and thereupon providing information, data, and/or instructions that could be utilized by the monitoring and/or treating syringe mechanism 100 using a variety of user-interface techniques, communication techniques, and/or networking techniques. For example, a remotely situated physician or other medical personnel could provide information or instructions indicating if the monitoring and/or treating syringe mechanism 100 should operate by applying, or not applying, the at least one fluid(s) and/or element(s) to the individual. For example, in certain embodiments, the monitoring and/or treating syringe mechanism 100 can inject the modified contents and the at least one fluid(s) and/or element(s) into the individual based at least in part in certain instances on remote instructions.

With certain embodiments of the monitoring and/or treating syringe mechanism 100, the physician or other health-care individual can be remotely situated while providing and monitoring a suitable treatment and/or regimen to the individual. Therefore, by using certain embodiments of the monitoring and/or treating syringe mechanism 100, the individual does not have to take time out of their schedule to visit the doctor or a hospital or clinic, such as by ensuring the treatment or the regimen is being followed and/or considering how the individual's condition is changing or responding. Such treatment and/or regimen as provided by certain embodiments of the monitoring and/or treating syringe mechanism 100 can consider a condition of the individual, as well as a condition of the at least one fluid(s) and/or element(s) into consideration when providing the treatment and/or regimen. A considerable amount of the health-care monitoring and/or treatment can thereby be provided remotely of the physician or other health-care individual utilizing certain embodiments of the monitoring and/or treating syringe mechanism 100.

Certain embodiments of the monitoring and/or treating syringe mechanism 100 can be configured to remotely monitor and/or treat individuals in a manner that may comply with the regimen. Certain embodiments of the monitoring and/or treating can be provided by the monitoring and/or treating syringe mechanism 100 such as to be provided remotely from a physician, medical personnel, that has created a treatment and/or regimen for the individual. Certain embodiments of the treatment or regimen such as provided by certain embodiments of the monitoring and/or treating syringe mechanism 100 can be responsive to the condition of the individual, and/or the condition of their at least one fluid(s) and/or element(s).

There may be a variety of treatments, illnesses, injuries, etc. at which a considerable portion of the treatment and/or regimen involves monitoring a condition of the individual, a condition of the at least one fluid(s), and/or monitoring a condition of the at least one element(s) associated with or included in the at least one fluid(s) and/or element(s). For example, such treatments and/or regimens can be provided for but are not limited to such conditions, elements, or diseases as, e.g., diabetes, flu, colds, AIDS, cancer, heart treatment medicine, etc. In certain instances, it may be desirable to provide such treatments or regimens in remote locations from a clinic, hospital, doctor's office, etc. at which remote location the individual cannot be easily monitored using the monitoring or feedback techniques and/or mechanisms as described in this disclosure. In certain instances, travel by the individual to a traditional health-care provider may be sufficiently difficult, time-consuming, or expensive to limit the individual's participation in the treatment and/or regimen. Certain countries and/or regions exist at which the individuals (as a result perhaps of financial difficulty and/or lack of medical education) may not pursue suitable medical treatments and/or medical regimens. Theses aspects, as well as others, can explain why a considerable percentage of traditional treatments and/or regimens prescribed by doctors are not followed by the individuals or patients. Certain embodiments of the monitoring and/or treating syringe mechanism 100 may be configurable to provide limited or no fluid(s) and/or element(s) in those instances that it determines the individual doesn't need the regimen, such as if they are analyzed being “cured”, or at least within prescribed limits.

Certain embodiments of the monitoring and/or treating mechanism 100 may be particularly adaptable to emergency or accident situations. Certain emergency or accident situations may exist, such as hurricanes, landslides, volcanoes, etc. where access to such medical treatments and/or regimens (or medical personnel who can apply such medical treatments and/or regimens) may be difficult or impossible. Certain embodiments of the monitoring and/or treating syringe mechanism 100 can allow for providing a suitable monitored treatment and/or regimen that are situated at locations remote from medical personnel, or which would be difficult for medical personnel to reach (e.g., to particular illnesses, sicknesses, etc. such as may be prevalent in those conditions) such as in certain emergency or accident situations. Certain embodiments of the monitoring and/or treating syringe mechanism 100 can limit the necessity of the individual to travel to the health-care provider and/or physician. As such, certain embodiments of the monitoring and/or treating syringe mechanism 100 can be configured to monitor, analyze and/or treat conditions likely to be encountered by such victims of particular emergency or accident situations such as could be easily detected. The emergency situations could include, but are not to necessarily limited to: diseases, flu, epidemics, and/or other treatable diseases. The medical capabilities of the individuals applying these emergency-based embodiments of the monitoring and/or treating syringe mechanism 100 can be considerably less than that of other implants and/or syringes, and as such aid workers could in certain instances provide rapid reliable aid to emergency or accident victims.

Certain embodiments of the monitoring and/or treating syringe mechanism 100 may be configured to provide particular medical treatments, situations, or regimens. With certain embodiments of traditional treating regimens, for example, the individual being treated (e.g., a patient) can obtain a responsive treating regimen by visiting a hospital, clinic, doctor's office, etc. at which the condition of the individual, their at least one fluid(s) and/or element(s) can be monitored. For instance, a physician or other medical personnel can, in certain instances, prepare a treatment and/or regimen that can vary depending upon whether some condition of the individual remains within certain prescribed limits. In certain circumstances when the condition of the individual (and/or the condition of their at least one fluid(s) and/or element(s)) falls outside of the prescribed limits, then the treatment and/or regimen can be suitably altered. One challenge with certain embodiments of the traditional treatment and/or regimen is to monitor the condition of the individual, their at least one fluid(s) and/or element(s) such as to provide a feedback mechanism for the treatment and/or regimen based at least in part on the condition of the individual and/or their at least one fluid(s) and/or element(s). Certain embodiments of the feedback mechanism can thereby monitor how the individual is responding to a particular treatment or regimen, and/or the condition of the individual.

Additionally certain traditional medical practices is placing high pressures on providing many of such regimens, monitoring, and/or treatment remotely from physicians, medical personnel, etc. such as to be able to reduce associated medical costs and/or expenses. It is important to ensure that the treatment and/or regimen remains directed to the condition of the individual such as to potentially provide, or determine not to provide the at least one modified fluid(s) and/or element(s) at least partially in response to the analyzing or monitoring the at least one monitored fluid(s) and/or element(s). Additionally, it may require considerable time and/or expense on the part of the individual to pursue certain traditional treatments and/or regimens. For these reasons and others, numerous individuals that have been placed on a variety of the treatment and/or regimens may, for one reason or another, fall off their treatment and/or regimen as a result of such factors as the expenses, time associated with treatment, remoteness of treatment, and/or other such associated difficulties.

As such, this disclosure describes a number of embodiments of the monitoring and/or treating syringe mechanism 100 that can be configured to analyze an individual; monitor an individual with respect to their compliance to a treatment and/or regimen; and/or treat the individual appropriately depending upon their compliance and/or non-compliance to treatment and/or regimen. Certain embodiments of the monitoring and/or treating syringe mechanism can be designed, utilized, modified, and/or provided to suit a particular individual and/or treat for a particular condition. Within this disclosure, the term “condition” can mean, depending upon context: illness, sickness, injury, etc.

The monitoring and/or treating syringe mechanism 100 can be utilized in variety of ways to allow a physician, a medical technician, and/or another medical care-providing person to provide an adaptive treatment and/or regimen to the individual. In certain embodiments, the term “adaptive treatment and/or regimen” may be considered as a treatment and/or regimen that can be altered based at least in part on the monitored condition of the individual, the at least one fluid(s) associated with the individual, and/or the at least one element(s) associated with the at least one fluid(s) that is associated with the individual. Within this disclosure, such altering of the treatment or regimen can potentially be performed in the absence of the physician or other medical care-providing person. Such an active treatment and/or regimen may be considered as a treatment and/or regimen having a feedback mechanism that is based at least in part on the monitored condition of the individual, the at least one fluid(s) associated with the individual, and/or the at least one element(s) associated with the at least one fluid(s) that is associated with the individual.

Certain embodiments of the treating regimen that can involve a feedback mechanism can, for example, involve applying some treatment (or some level of treatment) to the individual during such treatment periods as when the monitored condition of the individual is within some prescribed range, the at least one fluid(s) associated with the individual is within some prescribed range, and/or the at least one element(s) associated with the at least one fluid(s) that is associated with the individual is within some prescribed range. Similarly, the treatment could involve not applying the treatment (or applying a different level of treatment) to the individual during such treatment periods as when the monitored condition of the individual is not within some prescribed range, the at least one fluid(s) associated with the individual is not within some prescribed range, and/or the at least one element(s) associated with the at least one fluid(s) that is associated with the individual is not within some prescribed range. These types of treating regimens involving a feedback mechanism can be applied, for example, to apply treatment only in instances the treatment may benefit the health or condition of the individual.

Certain embodiments of the monitoring and/or treating syringe mechanism 100 can also be provided with different levels of automation, operation, and/or control. The different levels of automation, operation, and/or control for certain embodiments of the monitoring and/or treating syringe mechanism 100 can vary depending at least partially upon the type of treatment, and/or the complexity of treating the condition, illness, sickness, etc. Certain embodiments of the feedback mechanism can be at least partially computer or controller based, while certain embodiments of the feedback mechanism can rely on input, interaction, or other processes from the individual and/or other humans. Certain embodiments of the monitoring and/or treating syringe mechanism can analyze some aspect of the at least one withdrawn or aspirated fluid(s) and/or element(s), and thereupon allow the user (physician, hospital or ambulance worker, medical technician, etc.) of the monitoring and/or treating syringe mechanism 100 to determine the appropriate action based on their knowledge and the analysis. By comparison, certain embodiments of the monitoring and/or treating syringe mechanism can be largely automated, such as to be able to automatically inject one or more fluids and/or element(s) based, at least in part, on the analysis of the at least one fluid(s) and/or element(s) (which may be analyzed, controlled, mixed, and/or combined), such that the analysis can also be automated. Automation can be useful on applying a variety of the monitoring and/or treating syringe mechanism 100 where the individual and/or phlebotomist is untrained or unskilled.

In certain embodiments, the at least one fluid(s) and/or element(s) that can be withdrawn or aspirated from or injected into the individual may be analyzed to determine some condition or aspect of the individual. Certain embodiments of the monitoring and/or treating syringe mechanism 100 can thereby monitor whether a condition of the at least one fluid(s) and/or element(s) is outside of some prescribed limit for treatment. In certain embodiments, the monitoring and/or treating syringe mechanism 100 can analyze the at least one fluid(s) and/or element(s), and thereby in certain embodiments determine how the at least one fluid(s) and/or element(s) should be modified and thereupon be added to the individual at least partially using the monitoring and/or treating syringe mechanism 100. The at least one fluid(s) and/or element(s) that can be contained in a variety of organs can be monitored and/or analyzed in a variety of manners to effect treatment therefore.

In certain embodiments, the monitoring and/or treating syringe mechanism 100 can modify, filter, and/or replace the at least one fluid(s) and/or element(s), and/or combinations thereof that have been withdrawn or aspirated from the individual. In certain embodiments, the monitoring and/or treating syringe mechanism can inject an entirely different fluid(s), element(s), or combination thereof from those withdrawn or aspirated. By comparison, in certain embodiments, the analyzing the at least one fluid(s) and/or element(s) can be used to determine an element that could be added to the at least one fluid(s) and/or element(s) in an effort to benefit the individual at least partially using the monitoring and/or treating syringe mechanism 100.

Certain embodiments of the monitoring and/or treating syringe mechanism 100, as described with respect to FIG. 1, can include the feedback mechanism 112 that can monitor the condition of the individual, and/or the at least one fluid(s) and/or element(s) of the individual, and provide a suitable treatment or regimen of treatment at least partially in response to the monitored condition. Certain embodiments of the feedback mechanism 112 can operate substantially automatically, such as to require limited or no input from the individual and/or person operating or administering the monitoring and/or treating syringe mechanism 100. Certain embodiments of the feedback mechanism 112, by comparison, can operate based at least in part upon user input (e.g., the input at least partially derived from the individual operating or administering the monitoring and/or treating syringe mechanism 100).

Certain embodiments of the feedback mechanism 112 can thereby be configured to provide a treatment and/or regimen that is at least partially responsive to the monitored condition of the individual, the at least one fluid(s) and/or element(s). As such, certain embodiments of dispensing as at least partially perform by the treating and/or dispensing portion 94 can utilize the feedback mechanism 112 to act at least partially in response to the monitoring and/or analyzing as performed by the monitoring and/or analyzing portion 92, as described in this disclosure.

Certain embodiments of the monitoring and/or treating syringe mechanism 100 can thereby be configurable to monitor the individual, the at least one fluid(s) and/or element(s). Certain embodiments of the monitoring and/or treating syringe mechanism 100 can be configured to maintain and/or change one or more levels of one or more element(s) with respect to the at least one fluid(s) and/or element(s) that can be maintained and/or changed within an individual.

Certain embodiments of the transfer portion 102 of the monitoring and/or treating syringe mechanism 100 can be utilized to transfer the at least one fluid(s) and/or element(s), etc. between the individual and the monitoring and/or treating syringe mechanism 100. In certain instances, the particular type of the transfer portion 102 can largely dictate what the monitoring and/or treating syringe mechanism 100 can be used for. For instance, those embodiments of the transfer portion 102 that include patches may preferably be used to primarily transfer the at least one fluid(s) and/or element(s) from the monitoring and/or treating syringe mechanism 100 to the individual.

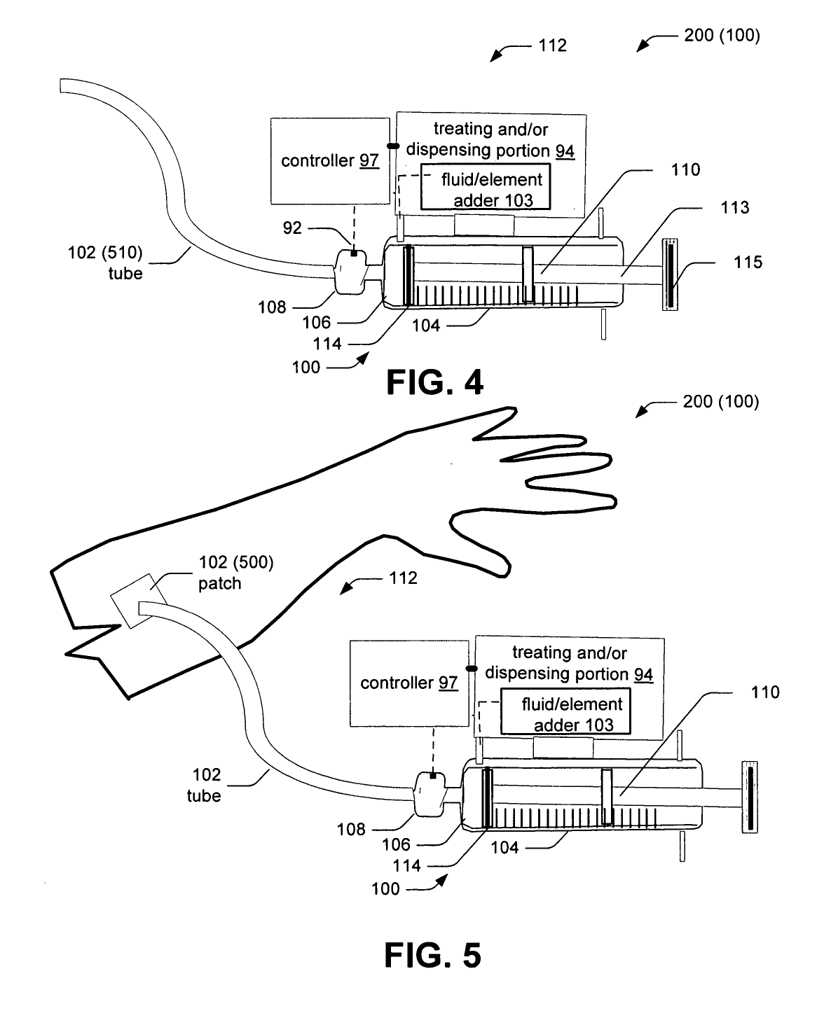

In certain instances, certain embodiments of the transfer portion 102 can be configured as to include one or more of the injecting-probe, the implant, the patch, the tube, and/or any other device that can transfer the at least one fluid(s) and/or element(s), etc. between the individual and the monitoring and/or treating syringe mechanism 100. Certain embodiments of the monitoring and/or treating syringe mechanism 100, as described with respect to FIG. 1, can be configured as the monitoring and/or treating syringe 200 as described with respect to FIGS. 2 or 5. The embodiments of the monitoring and/or treating syringe mechanism 100 (which may be an injecting-probe-based embodiment 200), as described with respect to FIG. 2, can include the injecting-probe as a transfer portion 102 that can be utilize to transfer such fluid(s) as liquid, perhaps mixed with at least one element(s). The embodiments of the monitoring and/or treating syringe mechanism 100 as described with respect to FIGS. 4 and 5, can include a tubular section that could be utilized to transfer such fluid(s) as gasses, perhaps mixed with at least one element(s) as well as liquids. Certain embodiments of the monitoring and/or treating syringe 200 can include, but is not limited to, a transfer portion 102, a fluid/element adder 103, a syringe barrel 104, a proximal chamber 106, the monitoring and/or treating controller 97, and/or a sheath 108 in fluid communication with the syringe barrel, and/or a plunger 110. Certain embodiments of the plunger 110 can include but are not limited to, e.g., a grip 15, a plunger rod 113, and a seal 114, the seal configurable to form a seal with the proximal chamber 106. In certain embodiments, the transfer portion 102 can be secured to the syringe barrel 104, and can be in fluid communication therewith. In certain embodiments, the transfer can be in the direction from the individual to the monitoring and/or treating syringe mechanism 100. In other embodiments, the transfer can be made in the direction from the monitoring and/or treating mechanism 100 to the individual. In certain embodiments, the at least one fluid(s) and/or element(s) can be configured primarily as a liquid; in other embodiments the at least one fluid(s) and/or element(s) can be configured primarily as a gas. The particular configuration of the at least one fluid(s) and/or element(s) can in certain instances affect the shape, size, configuration, or other design aspect of the transfer portion 102.

Certain embodiments of the fluid/element adder 103 of the monitoring and/or treating syringe mechanism 100 can be configured to add the at least one fluids, elements, components, chemicals, derivatives, heat, coolness, etc. to the at least one fluid(s) and/or element(s) that have been withdrawn or aspirated from the individual using the monitoring and/or treating syringe mechanism. Thereupon, in certain embodiments, the at least one fluid(s) and/or element(s) obtained from the fluid/element adder 103 can be combined with the at least one fluid(s) and/or element(s) that have been obtained from the individual to form at least one modified fluid and/or element that can be injected into the individual to afford certain embodiments of the treatment and/or regimen. For example, the embodiment of the monitoring and/or treating syringe mechanism 100 as described with respect to FIG. 2 including the embodiments of the injecting probe 102 as described with respect to FIGS. 2, 4, 5, 8, 9, 10, or 11 can be configured to inject or apply the same fluid(s) and/or element(s) (plus additional fluid(s) or element(s) that can be provided by the fluid/element adder 103) into the individual.

Certain embodiments of the fluid/element adder 103 of the monitoring and/or treating syringe mechanism can be configured to add one more fluids, elements, components, chemicals, derivatives, heat, coolness, etc. to the individual based at least in part on monitoring of the individual (or their fluid(s) and/or element(s)) to afford certain embodiments of the treatment. As such, the fluid(s) and/or element(s) being withdrawn from the individual can be analyzed, and based at least in part on the analysis, a completely different fluid(s) an/or element(s) may be added to the individual (or alternately depending on the condition of the at least one fluid(s) and/or element(s) as described in this disclosure, no fluid(s) or element(s)_may be added). As such, certain embodiments of the fluid/element adder 103 can be configured differently depending upon whether the at least one fluid(s) and/or element(s) being added to the individual is to be combined with the at least one withdrawn or aspirated fluid(s) and/or element(s) from the individual, or whether the at least one fluid(s) and/or element(s) is to be directly applied to the individual, or combined with some other at least one fluid(s) and/or element(s) to yield the at least one modified fluid(s) and/or element(s).

Certain embodiments of the syringe barrel 104 of the monitoring and/or treating syringe mechanism 100 as described with respect to FIGS. 2, 4, and 5 can be configured to contain, maintain, change, mix, process, and/or perform other operations on the at least one fluid(s) and/or element(s) that have either been withdrawn or aspirated from the individual and/or that have been provided at least in part by the fluid/element adder 103 as described in this disclosure. The configuration and/or usage of certain embodiments of the syringe barrel 104 are generally understood as providing an internal-sliding contact with the plunger 110 to at least partially define the proximal chamber 106 (which can change in dimension as a result of motion of the plunger), and will not be described further in this disclosure.

Certain embodiments of the proximal chamber 106 and/or the sheath 108 of the monitoring and/or treating syringe mechanism 100 as described with respect to FIG. 1 can be configured with the monitoring and/or treating controller 97. For example, the monitoring and/or treating controller 97 can be configured to analyze the at least one fluids and/or element(s) that have been withdrawn from the individual with the monitoring and/or treating syringe mechanism. Thereupon, depending upon the analysis, certain embodiments of the fluid/element adder 103 can add the at least one added fluid(s) and/or element(s) to the at least one withdrawn fluids and/or elements to yield the at least one modified fluids and/or elements. In certain embodiments, the at least one modified fluids and/or elements can be combined and/or mixed utilizing some combining or mixing mechanism that is not illustrated but is generally understood, e.g., a mixer of a suitable configuration situated within the syringe barrel 104. In certain embodiments, the at least one modified fluids and/or elements can be injected back into the individual.

Certain embodiments of the monitoring and/or treating controller 97 of the monitoring and/or treating syringe mechanism 100 as described with respect to FIGS. 1, 2, 4, and 5 can be configured to analyze and/or monitor the at least one fluid(s) and/or element(s) contained within the syringe barrel 104. Such monitoring and/or analyzing can be performed on the at least one fluid(s) and/or element(s) as withdrawn or aspirated from the individual, as provided by the fluid/element adder 103 or alternately as any combination thereof.

Certain embodiments of the sheath 108 of the monitoring and/or treating syringe mechanism 100 can be in fluid communication with at least a part of the syringe barrel, as described with respect to FIGS. 2, 4, and 5. Certain embodiments of the sheath 108 can be configured to assist in the withdrawal and/or injection with the monitoring and/or treating syringe mechanism 100. For instance, certain embodiments of the sheath 108 can be configured to decrease dead space for air to form within the monitoring and/or treating syringe mechanism, thereby limiting the possibility of a transmission of air bubbles to within the blood stream of the individual (which might, e.g., create an embolism, an inconsistent mixture, or another potentially dangerous or undesirable situation). Certain embodiments of the sheath 108 can also be configured as to limit the use of injecting-probe 102 reuse or needle-reuse. The configuration and use of a number of embodiments of the sheath 108 are generally understood, and will not be described further in this disclosure.

Certain embodiments of the plunger 110 of the monitoring and/or treating syringe mechanism 100 as described with respect to FIGS. 2, 4, or 5 can be configured to, upon actuation and/or displacement, aspirate, inject, and/or withdraw the at least one fluid(s) and/or element(s) between the monitoring and/or treating syringe mechanism 100 and the individual. Certain embodiments of the plunger 110 can be configured with a seal that can limit leakage of the at least one fluid(s) and/or element(s) between the plunger 110 and the syringe barrel 104. As such, in certain embodiments, the plunger 110 in combination with the syringe barrel 104 can at least partially form an enclosure for the at least one fluid(s) and/or element(s), wherein the at least one fluid(s) and/or element(s) can escape via the transfer portion 102 upon suitable actuation of the plunger 110 (e.g. by the individual 120 and/or the phlebotomist).

Certain embodiments of the monitoring and/or treating controller 97 as described with respect to FIGS. 1, 2, 4, or 5 can be utilized to sense, analyze, and/or monitor the at least one fluid(s) and/or element(s) that can be withdrawn or aspirated from the individual at least partially by the monitoring and/or treating syringe mechanism 100. Certain embodiments of the transfer portion 102 as described with respect to FIG. 2 can include, for example, the injecting-probe 102 that can be configured to withdraw, aspirate, and/or inject, the at least one fluid(s) and/or element(s) that can include, but is not limited to: a liquid, blood, saline, water, a combination of fluids, etc. In certain embodiments, the at least one fluid(s) and/or element(s) can be removed from different locations of the body of the individual. For example, certain embodiments of the monitoring and/or treating syringe mechanism 100 can withdraw blood from and/or inject blood into the individual's heart, the individual's aorta, the individual's arteries, the individual's veins, and/or certain of the individual's organs or capillaries. Certain aspects of the withdrawn or aspirated fluid(s) and/or element(s) such as blood may be dependent upon the location where the at least one fluid(s) and/or element(s) was withdrawn or aspirated. For example, oxygenated blood may be desired to be withdrawn or aspirated from, and/or could also be returned to the arteries, the aorta, certain portions of the heart, etc.; while de-oxygenated blood may be desired to be withdrawn or aspirated from, and could be returned to veins, etc.

The sophistication and/or complexity of the monitoring and/or treating syringe mechanism can, in many aspects, relate to certain aspects of the monitoring and/or treating controller 97. Certain embodiments of the monitoring and/or treating syringe mechanism 100 can perform one or more of the following operations entirely within a clean or sterile environment: withdrawing or aspirating the at least one fluid(s) and/or element(s) from the individual 120, analyzing the at least one fluid(s) and/or element(s), combining the at least one fluid(s) and/or element(s) with one or more fluid(s) and/or element(s) to form at least one modified fluid(s) and/or element(s), and/or injecting the at least one modified fluid(s) and/or element(s) into the individual 120. One aspect of the monitoring and/or treating syringe mechanism is that the at least one fluid(s) and/or element(s), as well as the at least one modified fluid(s) and/or element(s), can be maintained and/or changed in a clean or sterilized condition within the monitoring and/or treating syringe mechanism during this process.

A variety of applications for the monitoring and/or treating syringe mechanism 100 may be provided. For instance, FIG. 2 shows one application of the monitoring and/or treating syringe mechanism 100 that includes the injecting-probe which can be utilized to withdraw, aspirate, and/or inject liquids blood from the individual, analyze the blood, add suitable elements to the blood to derive modified blood, and return the modified blood to the individual. The embodiment of the transfer portion 102, which can in certain embodiments include the injecting-probe, can be used to inject and/or withdraw the at least one fluid(s) and/or element(s) at a variety of locations. For example, certain embodiments of the needle can inject and/or withdraw the at least one fluid(s) and/or element(s) with respect to arteries, veins, the aorta, the heart, and other embodiments of the body where blood, or other liquids that can be monitored, can be situated. During certain medical treatments or regimens, certain embodiments of the monitoring and/or treating syringe mechanism 100 can be applied to a particular suitable or desired location, organ, or system as generally understood in phlebotomy. Thereupon, a desirable quantity of liquid (e.g., blood) can be removed, and the removed liquid can be analyzed to determine which element(s) can be inserted in the fluid(s) and/or element(s).

Upon the combination, mixing, or insertion of the fluid(s) and/or element(s) into the at least one fluid(s) and/or element(s) to produce the at least one modified fluid(s) and/or element(s), the at least one modified fluid(s) and/or element(s) can thereupon be injected using certain embodiments of the monitoring and/or treating syringe mechanism 100 back into the individual. In certain embodiments, the site at which the at least one modified fluid(s) and/or element(s) is injected can be the same site at which the at least one fluid(s) and/or element(s) was originally removed (drawn) from, the individual. As such, the number of withdrawal and/or injection sites can be reduced as a result of using one or more sites for one or more processes. Reducing the number of injection sites for a particular treatment and/or regimen can make the treatment and/or regimen easier, less painful, and in certain instances more reliable. Certain regimens, as for cancer, chemotherapy, diabetes, AIDS, etc. rely on a number of injection sites for injections and/or withdrawals of the fluid(s) and/or element(s). In certain instances, certain individuals having low blood pressure, poor blood circulation, etc. may greatly appreciate the reduced number of injection sites. Additionally, there are a number of individuals who find injections and/or withdrawals to be painful or even make them queasy, who will appreciate the reduction of the number of injection sites by certain embodiments of the monitoring and/or treating syringe mechanism particularly desirable.

Another additional aspect of the monitoring and/or treating syringe mechanism 100 is that it can perform one or more of the operations (e.g., withdrawing, adding to, mixing, injecting) entirely within a clean or sterile environment as afforded by the monitoring and/or treating syringe mechanism. For example, the monitoring and/or treating syringe mechanism can withdraw or aspirate the at least one fluid(s) and/or element(s) from the individual 120, analyze the at least one fluid(s) and/or element(s), combine the at least one fluid(s) and/or element(s) with one more fluid(s) and/or element(s) to form the at least one modified fluid(s) and/or element(s), and/or inject the at least one modified fluid(s) and/or element(s) into the individual 120 in a manner that can utilize only one injection shot or hole from the monitoring and/or treating syringe mechanism while not allowing impurities to enter the at least one fluid(s) and/or element(s). Considering the large number of injecting-probe apertures (e.g. needle holes) certain medical treatments and/or regimens can require can make a number of such traditional treatments or regimens difficult, painful, potentially dangerous, and/or time consuming. By comparison, by certain embodiments of the monitoring and/or treating syringe mechanism reducing the number of injecting-probe shots or needle shots to provide the treatment and/or regimen can represent an improvement of time, pain, or danger to the individual as well as the medical care provider (e.g., the phlebotomist). As such, by controlling, changing, and/or maintaining certain aspects of the environment of certain embodiments of the monitoring and/or treating syringe mechanism, the environment that certain fluid(s) and/or element(s) being withdrawn, aspirating, and/or injected using the monitoring and/or treating syringe mechanism can be controlled, changed, and/or maintained as well.

Certain embodiments of the monitoring and/or treating syringe mechanism 100 may be included with a tube 5 10 as the transfer portion 102 that can be used to remove and/or inject gases such as air, gaseous medication, gaseous anesthesia, other gasses, and/or combinations of gasses as described with respect to FIG. 4. Certain embodiments of the tube 510 can be configured to transfer the at least one fluid(s) and/or element(s), for example, the tube 510 can be configured to pass or extend into the individual's mouth, nasal passage, an incision in the throat, etc. to allow the transfer of gasses between the individual and the monitoring and/or treating syringe mechanism. As such, certain embodiments of the transfer portion 102 can include, for example, the tube 510 that can be configured to withdraw, aspirate, or inject a gas such as air, oxygen, gaseous anesthesia, gaseous medications, a combination of gases, etc.

Certain embodiments of the monitoring and/or treating syringe mechanism 100 can include an implant portion 300, and/or a patch portion 500; and can act, for example, as to include the sensor 115 which can be configured to analyze the fluid(s) and/or element(s) such as air, which may be analyzed within or as withdrawn from the individual's throat, mouth, nasal passage, lungs, etc., and determining the percentage of carbon dioxide in the air. Thereupon, certain embodiments of the treating and/or dispensing portion of the monitoring and/or treating syringe mechanism 100 can apply oxygen via a suitable pathway such as the mouth, nasal passage, incision in the larynx, lungs, etc. Certain embodiments of the monitoring and/or treating syringe 200 can be configured to apply oxygen or other gasses to the lungs can be applied to individuals who are in a nursing home, hospital, emergency room, ambulance, etc.

Certain embodiments of one or more of the monitoring and/or treating syringe mechanism 100 can remove oxygen-depleted blood from at least one vein, oxygenate the blood, and return the blood to the arteries thereby bypassing the lungs. During a period of lung functional reduction or inoperability, certain embodiments of the monitoring and/or treating syringe mechanism 100 could even act to bypass and/or assist the operation of the lungs. With these embodiments of the monitoring and/or treating syringe mechanism 100, the analysis of certain gasses (e.g., O₂, CO₂, N, etc.) entering the monitoring and/or treating syringe mechanism can be significant in maintaining and/or changing the blood at suitable gas levels, as well as monitoring the condition of the individual and the blood. The sophistication or complexity of the monitoring and/or treating syringe 200 can be configured or designed depending upon its intended use, for example as a temporary life-maintaining system, as a longer-term maintaining system, or as a monitoring system remotely-situated from medical care.

Certain embodiments of the monitoring and/or treating syringe mechanism 100 may also be used to administer anesthesia. For example, the level of an anesthesia within an individual may be desired to be varied depending upon the level of the at least one fluid(s) and/or element(s) of the individual (e.g., carbon dioxide in the individual's lungs, blood, etc.). As such, certain embodiments of the monitoring and/or treating syringe mechanism 100 can sense whether the level of a particular one or more of the at least one fluid(s) and/or element(s) falls outside of some prescribed limit. Depending upon the actual monitored or analyzed range of such at least one fluid(s) and/or element(s), the at least one modified fluid(s) and/or element(s) being applied to the individual may be varied from what is typically applied. In certain instances, if the actual monitored or analyzed range of the at least one fluid(s) and/or element(s) fall within some prescribed (e.g., “normal”) range, then the treating and/or dispensing portion can dispense a normal level (or even simply maintain and/or change the condition of the fluid(s) and/or element(s)) of the at least one modified fluid(s) and/or element(s) to that individual to maintain and/or change medication and/or anesthesiology to the individual. By comparison, if the actual monitored or analyzed range of the at least one fluid(s) and/or element(s) fall outside of the prescribed (e.g., “normal”) range, then the treating and/or dispensing portion can dispense an altered level of the at least one modified fluid(s) and/or element(s) to that individual to provide an altered medication and/or an anesthesia to the individual. As such, certain embodiments of the monitoring and/or treating syringe mechanism 100 can be utilized to provide adaptive medication and/or adaptive anesthesia. Such adaptive medication and/or adaptive anesthesia can be controlled and/or regulated utilizing the feedback mechanism as described with respect to FIG. 1. Such adaptive medication and/or anesthesia can be applied whether the individual is conscious, unconscious, or medicated. Certain embodiments of the monitoring and/or treating syringe mechanism can thereby provide an active version of the feedback mechanism.

Certain embodiments of the monitoring and/or treating syringe mechanism 100 may not actually have to withdraw the at least one fluid(s) and/or element(s) from the individual to yield the at least one modified fluid(s) and/or element(s). For example, certain embodiments of the monitoring and/or treating syringe mechanism 100 can involve a variety of sensing and/or analyzing technology for the sensor 115 as described herein, certain ones of which may rely at least in part on wireless communications. Consider the embodiment of the implant-based sensor 115 as described with respect to FIG. 3, which can be configured as the mote, sensor device, controller device, computer-based device, or other mechanism that can be determined the condition of the at least one fluid(s) and/or element(s) within the individual 120. Thereupon, in certain embodiments, if some fluid or element should be injected and/or withdrawn from the individual, a communication can be established from the implant-based sensor 115 to another embodiment of the monitoring and/or treating syringe mechanism 100. The communication can thereupon be utilized to indicate whether fluid(s) and/or element(s) should be injected or withdrawn into or from the individual embodiments of the monitoring and/or treating syringe mechanism as described with respect to FIGS. 1, 2, 4, or 5, or other derivatives therefrom. Thereby, certain embodiments of the monitoring and/or treating syringe mechanism 100 can include a portion within the individual and another portion outside of the individual which can in combination monitor or analyze the condition of the at least one fluid(s) and/or element(s), and depending thereupon treat the individual based upon the monitored or analyzed condition. Based at least in part on the sensed or analyzed results, certain embodiments of the monitoring and/or treating syringe mechanism 100 can inject the at least one fluid(s), element(s), medication(s), and/or anesthesia(s) into the individual at least partially utilizing the treating and/or dispensing portion 94, as described in this disclosure.

Certain embodiments of the implant portion 300 can be considered as an inorganic and/or an organic device based upon such factors as the fluid(s) and/or the element(s) being contained, stored, and/or dispensed therefrom, as well as the duration which the fluid(s) and/or the element(s) are likely to be contained there within, and where the implant portion 300 may be situated within the individual 120. For example, certain embodiments of the implant portion 300 that are designed to be relatively temporary may be configured to be organic, such as to be able to be attacked, degraded, or disintegrated by organisms within the body of the individual. By comparison, certain embodiments of the implant portion 300 that are designed to be relatively permanent may be configured as inorganic, such as to have longer device-lifespans since they may have more resistance against being attacked or degraded by organisms within the body of the individual.

Certain embodiments of the implant portion 300 can be configured to communicate with devices external to the individual such as certain embodiments of the monitoring and/or treating syringe mechanism that can be configured as described with respect to FIGS. 1, 2, 4, or 5. As such, in certain embodiments, the implant portion 300 can analyze to derive information about the condition of the at least one fluid(s) and/or element(s). Thereupon, based at least in part on the derived information about the condition of the at least one fluid(s) and/or element(s), the monitoring and/or treating syringe mechanism 100 illustrated in block form in FIG. 3 could inject the at least one modified fluid(s) and/or element(s), or alternately could decide not to inject any fluid(s) and/or element(s). As such, certain embodiments of the monitoring and/or treating syringe mechanism 100 can treat the individual based on analysis or derived information relating to the condition of the at least one fluid(s) and/or element(s) within the individual.

As described in this disclosure, certain embodiments of the treating and/or dispensing portion 94 can be configured to dispense the at least one fluid(s) and/or element(s), and/or some combination thereof from the monitoring and/or treating syringe mechanism 100 to the individual, as described in this disclosure. In certain embodiments of the monitoring and/or treating syringe mechanism 100, the at least one fluid(s) and/or element(s) may be derived from the individual, from the monitoring and/or analyzing portion 92 of the monitoring and/or treating syringe mechanism 100, from some alternate source, and/or from some combination thereof.

Certain embodiments of the monitoring and/or treating syringe mechanism thereby can detect an indication of (e.g., an extinction or other) maintaining and/or changing the medical treatment and/or a regimen. Certain embodiments, of the monitoring and/or treating syringe mechanism 100 can notify someone utilizing established networking, communication, and/or data transfer techniques and/or otherwise take some appropriate action such as to provide an alarm which may be observed by the individual and/or others. Certain embodiments of the monitoring and/or treating syringe mechanism 100 can additionally provide a co-delivery of a microorganism stock with a nutrient or other reagent as one embodiment of the at least one fluid(s) and/or element(s). Certain embodiments of the monitoring and/or treating syringe mechanism 100 can be tailored to monitor or manage flora in one or more of the pulmonary tract, the genito-urinary tract, the digestive tract, or some other system.

FIG. 3 shows one embodiment of the implant portion 300 that can be configured to monitor for or sense a condition of the at least one fluid(s) and/or element(s). The information and/or data obtained from the embodiment of the implant portion 300 as described with respect to FIG. 3 can in certain embodiments be transmitted to a remote location. In certain embodiments, the implant portion 300 as described with respect to FIG. 3 can be configured to transmit information relating to the at least one fluid(s) and/or element(s) to another location that may be external of the individual. Another embodiment of the monitoring and/or treating syringe mechanism 100 can utilize the received information relating to the at least one fluid(s) and/or element(s) to potentially provide some treatment, etc.

FIG. 5 shows one embodiment of the patch portion 500 in which certain embodiments can be configured to transfer the at least one fluid(s) and/or element(s) to the individual. Certain embodiments of the patch portion 500 can be configured primarily as a dispenser to interact with the monitoring and/or treating syringe mechanism 100 to allow the at least one fluid(s) and/or element(s) to flow from the individual perhaps to the patch portion 500, and thereby act at least in part as the treating and/or dispensing portion 94 as described with respect to FIG. 1. In certain embodiments, the patch portion 500 may be kept in contact with the individual for an extended duration, or alternately may be removed relatively quickly, depending upon the type of treatment and/or regimen. Certain embodiments of the monitoring and/or treating syringe mechanism can provide chemicals (e.g., for chemotherapy), radioactive isotopes (e.g., for radiation), light or other electromagnetic radiation (e.g., for laser ablation), or other embodiments of the element.

Certain embodiments of the patch portion 500 as described with respect to FIG. 4 can also include the monitoring and/or analyzing portion 92 as described with respect to FIG. 1. Certain embodiments of the patch portion can also include the sensor 115 as described with respect to FIG. 1, can thereby act at least in part as the monitoring and/or analyzing portion 92 as described with respect to FIG. 1 to sense at least one condition of the individual and/or the condition of the at least one fluid(s) and/or element(s) of the individual. Certain embodiments of the sensor 115 and/or the monitoring and/or analyzing portion can be at least partially situated in the individual (e.g., a suitable lumen such as the bloodstream, the gastrointestinal tract, the respiratory tract, etc.) in such a manner as to provide information about the condition of the at least one fluid(s) and/or element(s). Thereupon, certain embodiments of the treating and/or dispensing portion 94 could dispense a variety of the at least one fluid(s) and/or element(s) (or even dispense nothing) as appropriate based at least in part on the sensed condition of the individual. A variety of patches are commercially available and generally known by those skilled in the technology, and may be utilized as the patch portion 500.

FIG. 4 shows another embodiment of the syringe-based monitoring and/or treating syringe mechanism 200 that can be configured to transfer the at least one fluid(s) and/or element(s) (particularly those configured as gases) between the individual and the monitoring and/or treating syringe mechanism. Certain embodiments of the tubular syringe-based monitoring and/or treating syringe mechanism 200 can be flexible such as to assist insertion, while other embodiments can be relatively rigid. Certain embodiments of the syringe-based monitoring and/or treating syringe mechanism 200 can be configurable to interface with a variety of the patch portion 500 as described with respect to FIG. 5. For example, the patch portion 500 can be particularly located at a desired location such as to be able to dispense the at least one fluid(s) and/or element(s), such as might be used to treat the individual. For instance, consider the embodiment where the individual is being treated for throat cancer. Certain embodiments of the syringe-based monitoring and/or treating syringe mechanism 200 can position certain embodiments of the implant portion 300 at a desired location, such as the throat of the individual. Following implant, a variety of techniques can be provided to derive the relative position of the implant portion 300, and thereupon dispense the at least one fluid(s)/element(s) as appropriate for the particular illness or sickness. As such, cancer, heart disease, AIDS, and a variety of other illnesses or diseases can be suitably monitored and/or analyzed at the particular location of the illness or disease, and precisely targeted utilizing a number of embodiments of the monitoring and/or treating syringe mechanism 100. Certain embodiments of the syringe-based monitoring and/or treating syringe mechanism 200 can include the tubular segment 510 that can be configured to transfer gaseous or liquid embodiments of the at least one the fluid(s) and/or element(s). For example, certain embodiments of the tubular segment 510 can be inserted down the throat, nasal passage, larynx, etc. of the individual. As such, the embodiment of the syringe-based monitoring and/or treating syringe mechanism 200, as described with respect to FIG. 4, can be configured to monitor and/or analyze gasses.

Certain embodiments of the treating and/or dispensing portion 94 can be configured to dispense the at least one fluid(s) and/or element(s) based at least in part on feedback information derived by the feedback mechanism, as described in this disclosure with respect to FIG. 1. As such, in certain embodiments, analyzed information as derived at least in part from analysis of the at least one fluid(s) and/or element(s) that can be provided as treatment at least in part on the monitoring and/or analyzing portion 92.

Certain embodiments of the monitoring and/or treating syringe mechanism 100 can be configured with suitable valves as appropriate to provide for withdrawing and/or injecting the at least one fluid(s) and/or element(s). For example, for those embodiments of the monitoring and/or treating syringe mechanism 100 that are associated with the uniaxial needle as described with respect to FIG. 8, the fluid/element adder 103 could be valved to allow insertion of the fluid(s) and/or element(s) from the fluid/element adder 103 as described with respect to FIG. 1 to form the at least one modified fluid and/or element during the withdrawal stroke of the monitoring and/or treating syringe mechanism 100 but not during the injection stroke of the monitoring and/or treating syringe mechanism 100, or vice versa. A controller-actuated valve can accomplish a similar injection or combination of the at least one fluid(s) and/or element(s) from the fluid/element adder 103 during particular strokes. The use and design of the valves (including fluid-actuated valves, pneumatically actuated valves, and/or controller-actuated valves) would be known to those skilled in the valving and/or actuator technologies, and will not be further detailed herein.

For those embodiments of the injecting probe 102 that include multiple lumens, such as described with respect to FIGS. 9 or 10, one lumen can be devoted to the at least one fluid(s) and/or element(s) being expelled and injected into the individual, while the other lumen can be devoted with the at least one fluid(s) and/or element(s) being withdrawn from the individual. Each lumen of the multi-lumen embodiments of the injecting probe 102 can be fluidly associated with a distinct unidirectional valve. For example, the unidirectional valve lumen used to withdraw the at least one fluid(s) and/or element(s) can allow the fluids(s) and element(s) to flow out of the individual, but not towards the individual. By comparison, the unidirectional valve lumen used to inject the at least one fluid(s) and/or element(s) into the individual can allow the fluids(s) and element(s) to flow into the individual, but not out of the individual. As such, in certain embodiments, one lumen can act to withdraw the at least one fluid(s) and/or element(s), and another lumen can act to inject the at least one fluid(s) and/or element(s).

Certain embodiments of the monitoring and/or treating syringe mechanism 100 can be configured as described with respect to FIG. 12 to withdraw at least one fluid(s) and/or element(s) from the individual that can be thereupon discarded via open one-way valve V1 into the discard reservoir 1250. During this withdrawing process, the controller 97 can sense the characteristics of the fluid(s) and/or element(s) as contained within the individual. During the withdrawing process, the valve V2 can close to limit the flow of the at least one withdrawn fluid(s) and/or element(s) from the individual into the syringe barrel 104, and the withdraw stroke of the monitoring and/or treating syringe mechanism 100 can pull the at least one fluid(s) and/or element(s) from the fluid/element adder 103 into the proximal chamber 106.

During the injection stroke of the embodiment of the monitoring and/or treating syringe mechanism 100 as described with respect to FIG. 12, the one-way valve V2 is open to allow fluid to pass via the valve V2 and the injecting probe 102 to the individual 120. During the injection stroke, the valve V1 can be closed to limit suction of the fluid in the discard reservoir 1250 via the injecting probe 102 to the individual. As such, the embodiment of the monitoring and/or treating syringe mechanism 100 as described with respect to FIG. 12 can be configured to provide a virtually entirely different at least one fluid(s) and/or element(s) from the individual as compared to that withdrawn from the individual.

As such, certain embodiments of the monitoring and/or treating syringe mechanism 100 can be configured to monitor the condition of the at least one fluid(s) and/or element(s) in a variety of configurations, precisions, durations, etc. In certain embodiments, a physician can provide a variable treatment mechanism using certain embodiments of the monitoring and/or treating syringe mechanism 100 that can vary depending at least in part on the condition of the at least one fluid(s) and/or element(s) as withdrawn from and/or maintained within the individual.

### 2. CERTAIN EMBODIMENTS OF THE MONITORING AND/OR TREATING CONTROLLER

This disclosure describes a number of embodiments of the monitoring and/or treating controller as described with respect to FIG. 1 that are attended to control operations of the monitoring and/or treating syringe mechanism 100. Certain embodiments of the monitoring and/or treating controller can act as are provided the functionality of the at least a portion of the feedback mechanism as described with respect to FIG. 1.

FIG. 1 shows a block diagram of certain embodiments of the monitoring and/or treating syringe mechanism 100 that include the monitoring and/or treating controller 97. Certain embodiments of the monitoring and/or treating syringe mechanism 100 can include, but are not limited to, any particular configuration of the monitoring and/or treating controller 97. Certain embodiments of the monitoring and/or treating controller 97 can be computer based, mote based, and/or electronics based. Certain embodiments of the monitoring and/or treating controller can be segmented into modules, certain of which can be at least partially within the individual's body while others of which can be at least partially outside of the individual's body. Certain embodiments of the monitoring and/or treating controller 97 can utilize a variety of wireless communication and/or networking technology to allow information, data, etc. to be transferred to the various distinct portions of the monitoring and/or treating syringe mechanism 100.

Certain embodiments of the monitoring and/or treating controller 97 can vary as to their automation, complexity, and/or sophistication; and can be utilized to analyze the at least one fluid(s) and/or element(s) withdrawn or aspirated from and/or injected into the individual, control the at least one fluid(s) and/or element(s) added to the individual, and/or control the at least one fluid(s) and/or element(s) withdrawn, aspirated and/or fluid injected relative the individual. As described within this disclosure, multiple ones of the different embodiments of the monitoring and/or treating syringe mechanism 100 can transfer information about the individual 120 or their condition, information or data relating to the fluid(s) and/or element(s), signals, etc. via a communication link to or from a remote monitoring location and/or some intermediate device as might be associated with monitoring and/or other activities.

Certain embodiments of the monitoring and/or treating controller 97, as well as certain embodiments of the monitoring and/or treating syringe mechanism 100 in general, can utilize distinct firmware, hardware, and/or software technology. For example, mote-based technology, microprocessor-based technology, microcomputer-based technology, general-purpose computer technology, specific-purpose to computer technology, and a variety of other computer technologies can be utilized for certain embodiments of the monitoring and/or treating controller 97, as well as certain embodiments of the monitoring and/or treating syringe mechanism 100.

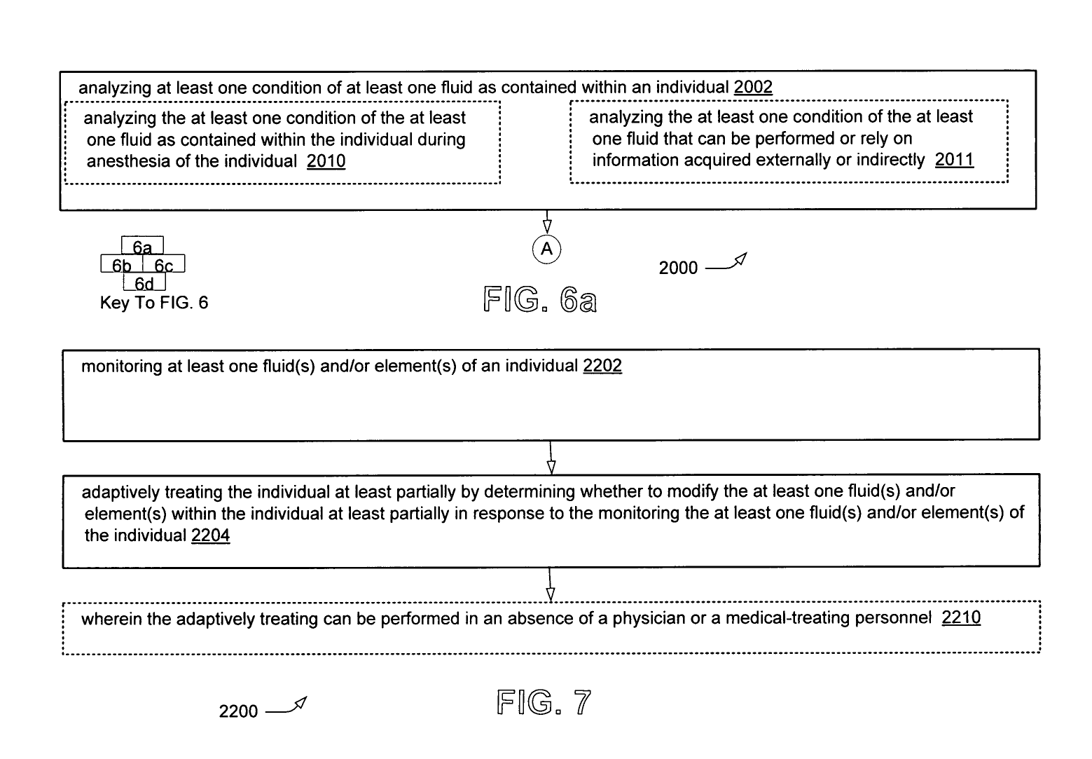

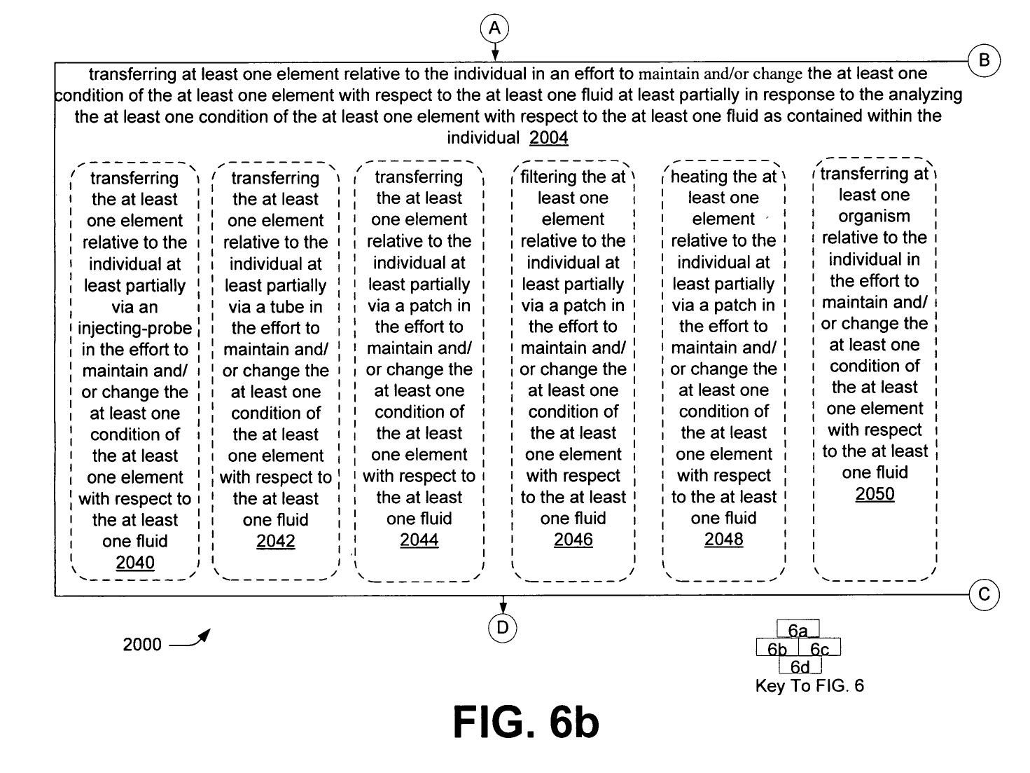

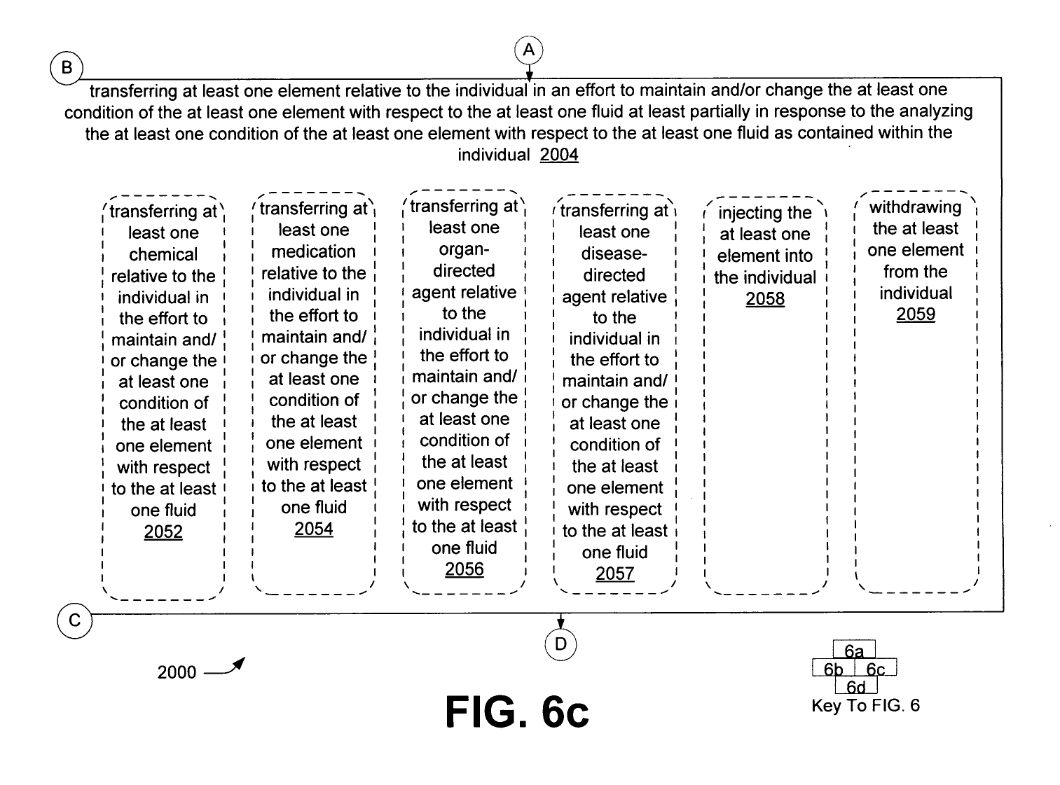

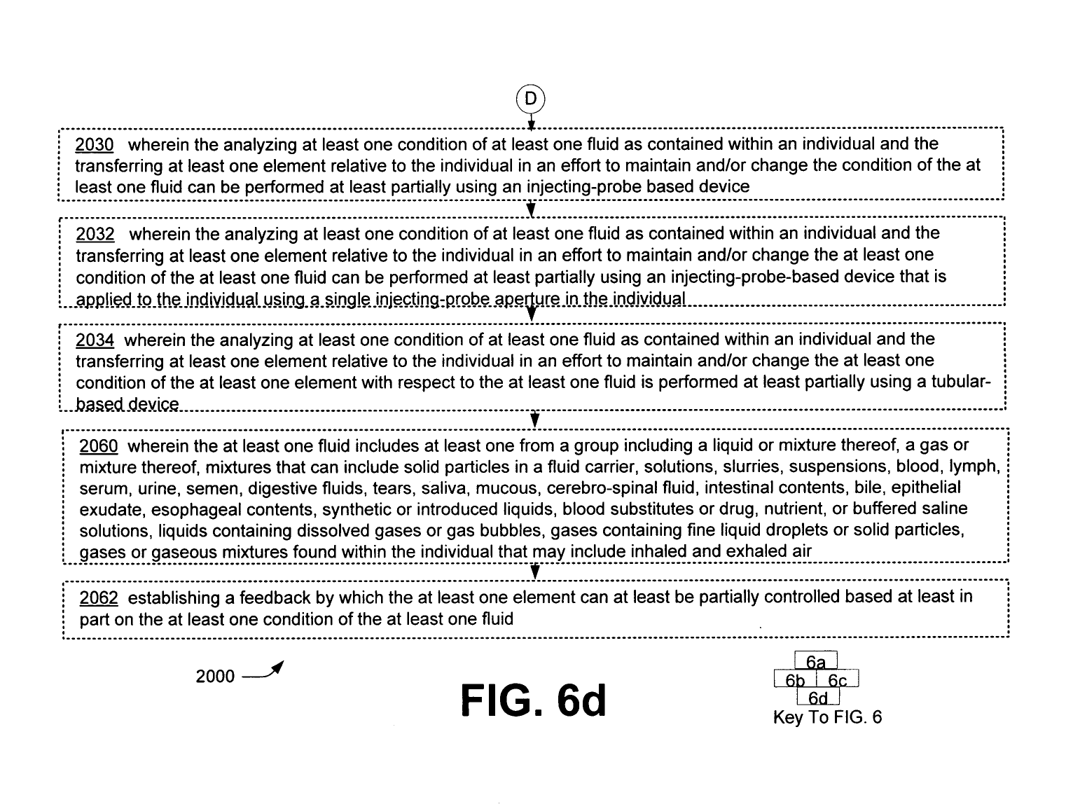

Certain embodiments of the monitoring and/or treating controller 97 can include a processor 803 such as a central processing unit (CPU), a memory 807, a circuit or circuit portion 809, and an input output interface (I/O) 811 that may include a bus (not shown). Certain embodiments of the monitoring and/or treating controller 97 of the monitoring and/or treating syringe mechanism 100 can include and/or be a portion of a general-purpose computer, a specific-purpose computer, a microprocessor, a microcontroller, a personal display assistant (PDA), a cellular phone, a wireless communication device, a hard-wired phone, and/or any other known suitable type of communications device, computer, and/or controller that can be implemented in hardware, software, electromechanical devices, and/or firmware. Certain portions of the monitoring and/or treating controller 97 of the monitoring and/or treating syringe mechanism 100 can utilize physically or operably configurable phlebotomy, monitoring, and/or other associated or unrelated activities. Certain embodiments of the processor 803, as described with respect to FIG. 6, can perform the processing and arithmetic operations for certain embodiments of the monitoring and/or treating controller 97 of the monitoring and/or treating syringe mechanism 100. For instance, dosing, rate of application, and certain other such aspects can be performed by the processor 803. Certain embodiments of the monitoring and/or treating controller 97 of the monitoring and/or treating syringe mechanism 100 can control the signal processing, database querying and response, computational, timing, data transfer, and other processes associated with certain embodiments of the monitoring and/or treating controller 97 of the monitoring and/or treating syringe mechanism 100.

Certain embodiments of the memory 807 of the monitoring and/or treating controller 97 can include a random access memory (RAM) and/or read only memory (ROM) that together can store the computer programs, operands, and other parameters that control the operation of certain embodiments of the monitoring and/or treating controller 97 of the monitoring and/or treating syringe mechanism 100. The memory 807 can be configurable to contain the defibrillation information or individual information obtained, retained, or captured by that particular monitoring and/or treating controller 97 of the monitoring and/or treating syringe mechanism 100.

Certain embodiments of the bus can be configurable to provide for digital information transmissions between the processor 803, circuits 809, memory 807, I/O 811, and/or the image memory or storage device (which may be integrated or removable). In this disclosure, the memory 807 can be configurable as RAM, flash memory, semiconductor-based memory, of any other type of memory that can be configurable to store data pertaining to images. The bus also connects I/O 811 to the portions of certain embodiments of the monitoring and/or treating controller 97 of the monitoring and/or treating syringe mechanism 100 that either receive digital information from, or transmit digital information to other portions of the monitoring and/or treating syringe mechanism 100.

Certain embodiments of the monitoring and/or treating controller 97 of the monitoring and/or treating syringe mechanism 100 as described with respect to FIG. 1 can include a transmitter portion (not shown) that can be either included as a portion of certain embodiments of the monitoring and/or treating controller 97 of the monitoring and/or treating syringe mechanism 100, or alternately can be provided as a separate unit (e.g., microprocessor-based). In certain embodiments, the transmitter portion can transmit image information between certain embodiments of the monitoring and/or treating controller 97 of the monitoring and/or treating syringe mechanism 100.

Certain embodiments of the monitoring and/or treating controller 97 of the monitoring and/or treating syringe mechanism 100 as described with respect to FIG. 1 can include an operation altering portion (not shown) that can be either included as a portion of certain embodiments of the monitoring and/or treating controller 97 of the monitoring and/or treating syringe mechanism 100, or alternately can be provided as a separate unit (e.g., microprocessor-based). Examples of operation altering portions include, but are not limited to, altering a resolution, altering a contextual library, altering an aspect ratio, altering a color intensity and/or brightness or particular monitoring and/or treating syringe mechanism 100.

Certain embodiments of the memory 807 can provide one example of a memory storage portion. In certain embodiments, the monitored value includes but is not limited to: a percentage of the memory 807, a number of images that are stored in the memory 807, or for data storage or recording interval (audio or video recording intervals).

To provide for overflow ability for the memory 807 of certain embodiments of the monitoring and/or treating controller 97 of the monitoring and/or treating syringe mechanism 100, the image storage device can be operably coupled to the memory 807 to allow a controllable transmitting of memory data from certain embodiments of the monitoring and/or treating controller 97 of the monitoring and/or treating syringe mechanism 100 when the monitored value of data within the memory 807 (e.g., the memory storage portion) exceeds a prescribed value. The prescribed value can include, e.g., some percentage amount or some actual amount of the value.

In certain embodiments, a secondary communication link can be established between the certain embodiments of the monitoring and/or treating controller 97 of the monitoring and/or treating syringe mechanism 100. The secondary communication link can be structured similar to as a communication link, or alternatively can utilize network-based computer connections, Internet connections, etc. to provide information and/or data transfer between certain embodiments of the monitoring and/or treating controller 97 of the monitoring and/or treating syringe mechanism 100. As such, certain embodiments of monitoring and/or treating syringe mechanism 100, a remotely-situated doctor, medical technician, nurse, medical analysis, etc. could analyze data or other such information transmitted from the monitoring and/or treating syringe mechanism, and thereupon analyze suitable operation of the monitoring and/or treating syringe mechanism based at least partially upon the analysis. In certain embodiments, the remotely-situated doctor, medical technician, nurse, medical analysis, etc. could communicate with a phlebotomist or other individual operating the monitoring and/or treating syringe mechanism 100.

In certain embodiments, the remotely-situated doctor, medical technician, nurse, medical analysis, etc. could respond to the information of data transmitted from the monitoring and/or treating syringe mechanism 100 with remote medical information, that in certain embodiments could somehow be applied to and/or utilized by the monitoring and/or treating syringe mechanism 100. For instance, certain embodiments of the remote medical information could include but is not limited to: monitoring and/or treating syringe mechanism operational information (e.g., information instructing the phlebotomist to, or not to, inject the contents of the monitoring and/or treating syringe mechanism into the individual), element adding instructions (e.g., add the following contents to the contents in the monitoring and/or treating syringe mechanism, heat the contents of the monitoring and/or treating syringe mechanism, etc.), etc. Certain embodiments of the remote medical information could be largely automated, or alternatively remain primarily up to the phlebotomist to apply. Additionally, a variety of communication devices, networking devices, computing devices, user interfaces, etc. could be utilized to enhance communications between a remotely-situated doctor, medical technician, nurse, medical analysis, etc. and the phlebotomist.

In certain embodiments of the monitoring and/or treating controller 97 of the monitoring and/or treating syringe mechanism 100, the particular elements of certain embodiments of the monitoring and/or treating controller 97 of the monitoring and/or treating syringe mechanism 100 (e.g., the processor 803, the memory 807, the circuits 809, and/or the I/O 811) can provide a monitoring function to monitor or sense at least one condition of the individual. A monitoring function as provided by certain embodiments of the monitoring and/or treating controller 97 of the monitoring and/or treating syringe mechanism 100 can be compared to a prescribed limit, such as whether the number of images contained in the memory 807, the amount of data contained within the memory 807, or some other measure relating to the memory is approaching some value. The limits to the value can, in different embodiments, be controlled by the user or the manufacturer of certain embodiments of the monitoring and/or treating controller 97 of the monitoring and/or treating syringe mechanism 100. In certain embodiments, the memory 807 can store such information as data, information, displayable information, readable text, motion images, video images, and/or audio images, etc.

In certain embodiments, the I/O 811 provides an interface to control the transmissions of digital information between each of the components in certain embodiments of the monitoring and/or treating controller 97 of the monitoring and/or treating syringe mechanism 100. The I/O 811 also provides an interface between the components of certain embodiments of the monitoring and/or treating controller 97 of the monitoring and/or treating syringe mechanism 100. The circuits 809 can include such other user interface devices as a display and/or a keyboard.

In other embodiments, the monitoring and/or treating controller 97 of the monitoring and/or treating syringe mechanism 100 can be constructed as a specific-purpose computer such as an application-specific integrated circuit (ASIC), a microprocessor, a microcomputer, or other similar devices.

A variety of devices, locations, etc. could be configured as and/or include certain embodiments of the monitoring and/or treating syringe mechanism 100. For example, a portion of an automobile or ambulance (either internal or external location thereof) could be provided with certain embodiments of the monitoring and/or treating syringe mechanism. A trained dog (such as used in ski patrols, etc.) could be fitted with certain embodiments of the monitoring and/or treating syringe mechanism 100, and allowed to run to those in need of emergency medical care. Hospitals, care centers, schools, ski patrols, life guards, workplaces, ambulance workers, forest workers, etc. could each be provided with their individualized version of the monitoring and/or treating syringe mechanism 100, as necessary. A variety of vehicles, seats, etc. can also be configured with certain embodiments of the monitoring and/or treating syringe mechanism 100. For instance, certain hospitals, rest homes, doctor offices, etc. can be provided with certain embodiments of the monitoring and/or treating syringe mechanism 100.

### 3. CERTAIN EMBODIMENTS OF MONITORING AND/OR TREATING SYRINGE MECHANISM WITH RELEVANT FLOWCHARTS

Within the disclosure, flow charts of the type described in this disclosure apply to method steps as performed by a computer or controller. The flow charts can also apply to apparatus devices, such as an antenna or a node associated therewith that can include, e.g., a general-purpose computer or specialized-purpose computer whose structure along with the software, firmware, electromechanical devices, and/or hardware, can perform the process or technique described in the flow chart.

One embodiment of a high-level flowchart of a defibrillation technique 2000 is described with respect to FIG. 6 (including FIGS. 6a, 6b , 6c, and 6d) and can include, but is not limited to, operations 2002 and 2004, and optional operations 2030, 2032, 2034, 2060, and/or 2062. One embodiment of operation 2002 can include, but is not limited to, optional operations 2010 and 2011. One embodiment of operation 2004 can include, but is not limited to, optional operations 2040, 2042, 2044, 2046, 2048, 2050, 2052, 2054, 2056, 2057, 2058, and/or 2059. The high-level flowchart of FIG. 6 (including FIGS. 6a, 6b, 6c, and 6d) should be considered in combination with the embodiments of the monitoring and/or treating syringe mechanism 100, as described with respect to FIGS. 1 to 5. One embodiment of operation 2002 can include, but is not limited to, analyzing at least one condition of at least one fluid as contained within an individual. For example, one embodiment of the monitoring and/or treating syringe mechanism 100 as described in this disclosure can be configured to analyze the condition of the at least one fluid(s) and/or element(s) within the individual, or alternately as withdrawn from the individual. One embodiment of operation 2004 can include, but is not limited to, transferring at least one element relative to the individual in an effort to maintain and/or change the at least one condition of the at least one element with respect to the at least one fluid at least partially in response to the analyzing the at least one condition of the at least one fluid as contained within the individual. For example, one embodiment of the at least one modified fluid(s) and/or element(s) can be transferred either from the individual to the monitoring and/or treating syringe mechanism 100, or vice versa. One embodiment of the analyzing at least one condition of at least one fluid as contained within an individual of operation 2002 can include operation 2010, that can include, but is not limited to, analyzing the at least one condition of the at least one fluid as contained within the individual during anesthesia of the individual. For example, such fluids as blood or air could be analyzed when the individual is anesthetized, in certain instances to maintain and/or change the level of anesthesia in the individual. One embodiment of the analyzing at least one condition of at least one fluid as contained within an individual of operation 2002 can include operation 2011, that can include, but is not limited to, analyzing the at least one condition of the at least one fluid that can be performed or rely on information acquired externally or indirectly. For example, such fluids as blood or air could be analyzed based on information acquired indirectly, such as from an injected device such as a mote as described with respect to FIG. 2. One embodiment of operation 2030 can include, but is not limited to, wherein the analyzing at least one condition of at least one fluid as contained within an individual and the transferring at least one element relative to the individual in an effort to maintain and/or change the at least one condition of the at least one fluid can be performed at least partially using an injecting-probe-based device. For example, certain embodiments of the monitoring and/or treating syringe mechanism 100 as described in this disclosure can utilize the injecting-probe. One embodiment of operation 2032 can include, but is not limited to, wherein the analyzing at least one condition of at least one fluid as contained within an individual and the transferring at least one element relative to the individual in an effort to maintain and/or change the at least one condition of the at least one fluid can be performed at least partially using an injecting-probe-based device that is applied to the individual using a single injecting-probe aperture in the individual. For example, certain embodiments of the monitoring and/or treating syringe mechanism 100 as described in this disclosure that can perform the analyzing can utilize the injecting-probe as injected through a single injecting-probe aperture in the individual. One embodiment of operation 2034 can include, but is not limited to, wherein the analyzing at least one condition of at least one fluid as contained within an individual and the transferring at least one element relative to the individual in an effort to maintain and/or change the at least one condition of the at least one element with respect to the at least one fluid is performed at least partially using a tubular-based device. For example, certain embodiments of monitoring and/or treating syringe mechanism 100 as described in this disclosure can include a tube, certain embodiments which could be inserted in the individual's mouth, nose, or larynx. One embodiment of the transferring at least one element relative to the individual in an effort to maintain and/or change the at least one condition of the at least one element with respect to the at least one fluid at least partially in response to the analyzing the at least one condition of the at least one fluid as contained within the individual of operation 2004 can include operation 2040, that can include, but is not limited to, transferring the at least one element relative to the individual at least partially via an injecting-probe in the effort to maintain and/or change the at least one condition of the at least one element with respect to the at least one fluid. For example, the injecting-probe 102 can be used to transfer the element relative to the individual. One embodiment of the transferring at least one element relative to the individual in an effort to maintain and/or change the at least one condition of the at least one element with respect to the at least one fluid at least partially in response to the analyzing the at least one condition of the at least one fluid as contained within the individual of operation 2004 can include operation 2042, that can include, but is not limited to, transferring the at least one element relative to the individual at least partially via a tube in the effort to maintain and/or change the at least one condition of the at least one element with respect to the at least one fluid. For example, the tube can be used to transfer the element relative to the individual. One embodiment of the transferring at least one element relative to the individual in an effort to maintain and/or change the at least one condition of the at least one element with respect to the at least one fluid at least partially in response to the analyzing the at least one condition of the at least one fluid as contained within the individual of operation 2004 can include operation 2044, that can include, but is not limited to, transferring the at least one element relative to the individual at least partially via a patch in the effort to maintain and/or change the at least one condition of the at least one element with respect to the at least one fluid. For example, the patch can be used to transfer the element relative to the individual. One embodiment of the transferring at least one element relative to the individual in an effort to maintain and/or change the at least one condition of the at least one element with respect to the at least one fluid at least partially in response to the analyzing the at least one condition of the at least one fluid as contained within the individual of operation 2004 can include operation 2046, that can include, but is not limited to, filtering the at least one element relative to the individual at least partially via a patch in the effort to maintain and/or change the at least one condition of the at least one element with respect to the at least one fluid. For example, the at least one fluid(s) and/or element(s) can be filtered to filter at least some of the element. One embodiment of the transferring at least one element relative to the individual in an effort to maintain and/or change the at least one condition of the at least one element with respect to the at least one fluid at least partially in response to the analyzing the at least one condition of the at least one fluid as contained within the individual of operation 2004 can include operation 2048, that can include, but is not limited to, heating the at least one element relative to the individual at least partially via a patch in the effort to maintain and/or change the at least one condition of the at least one element with respect to the at least one fluid. For example, the at least one fluid(s) and/or element(s) can be heated. One embodiment of the transferring at least one element relative to the individual in an effort to maintain and/or change the at least one condition of the at least one element with respect to the at least one fluid at least partially in response to the analyzing the at least one condition of the at least one fluid as contained within the individual of operation 2004 can include operation 2050, that can include, but is not limited to, transferring at least one organism relative to the individual in the effort to maintain and/or change the at least one condition of the at least one element with respect to the at least one fluid. For example, certain embodiments of a living, or not living, organism can be transferred relative to the individual. One embodiment of the transferring at least one element relative to the individual in an effort to maintain and/or change the at least one condition of the at least one element with respect to the at least one fluid at least partially in response to the analyzing the at least one condition of the at least one fluid as contained within the individual of operation 2004 can include operation 2052, that can include, but is not limited to, transferring at least one chemical relative to the individual in the effort to maintain and/or change the at least one condition of the at least one element with respect to the at least one fluid. For example, certain embodiments of the at least one chemical can be transferred relative to the individual to maintain and/or change the at least one condition of the element. One embodiment of the transferring at least one element relative to the individual in an effort to maintain and/or change the at least one condition of the at least one element with respect to the at least one fluid at least partially in response to the analyzing the at least one condition of the at least one fluid as contained within the individual of operation 2004 can include operation 2054, that can include, but is not limited to, transferring at least one medication relative to the individual in the effort to maintain and/or change the at least one condition of the at least one element with respect to the at least one fluid. For example, at least one medication can be transferred relative to the individual. One embodiment of the transferring at least one element relative to the individual in an effort to maintain and/or change the at least one condition of the at least one element with respect to the at least one fluid at least partially in response to the analyzing the at least one condition of the at least one fluid as contained within the individual of operation 2004 can include operation 2056, that can include, but is not limited to, transferring at least one organ-directed agent relative to the individual in the effort to maintain and/or change the at least one condition of the at least one element with respect to the at least one fluid. For example, at least one agent that is directed to at least one organ can be transferred relative to the individual. One embodiment of the transferring at least one element relative to the individual in an effort to maintain and/or change the at least one condition of the at least one element with respect to the at least one fluid at least partially in response to the analyzing the at least one condition of the at least one fluid as contained within the individual of operation 2004 can include operation 2057, that can include, but is not limited to, transferring at least one disease-directed agent relative to the individual in the effort to maintain and/or change the at least one condition of the at least one element with respect to the at least one fluid. For example, at least one agent that is directed to at least one disease can be transferred relative to the individual. One embodiment of the transferring at least one element relative to the individual in an effort to maintain and/or change the at least one condition of the at least one element with respect to the at least one fluid at least partially in response to the analyzing the at least one condition of the at least one fluid as contained within the individual of operation 2004 can include operation 2058, that can include, but is not limited to, injecting the at least one element into the individual. For example, the at least one element can be injected into the individual. One embodiment of the transferring at least one element relative to the individual in an effort to maintain and/or change the at least one condition of the at least one element with respect to the at least one fluid at least partially in response to the analyzing the at least one condition of the at least one fluid as contained within the individual of operation 2004 can include operation 2059, that can include, but is not limited to, withdrawing the at least one element from the individual. For example, the at least one element can be withdrawn from the individual. One embodiment of operation 2060 can include, but is not limited to, wherein the at least one fluid includes at least one from a group including a liquid or mixture thereof, a gas or mixture thereof, mixtures that can include solid particles in a fluid carrier, solutions, slurries, suspensions, blood, lymph, serum, urine, semen, digestive fluids, tears, ambiotic fluid, amniotic fluid, saliva, mucous, cerebro-spinal fluid, intestinal contents, bile, epithelial exudate, esophageal contents, synthetic or introduced liquids, blood substitutes or drug, nutrient, or buffered saline solutions, liquids containing dissolved gases or gas bubbles, gases containing fine liquid droplets or solid particles, gases or gaseous mixtures found within the individual that may include inhaled and exhaled air. For example, the at least one fluid can include a least one from this group. One embodiment of operation 2062 can include, it is not limited to, establishing a feedback by which the at least one element can at least be partially controlled based at least in part on the at least one condition of the at least one fluid. For example, a feedback mechanism can be provided by which the element to be transferred can be controlled based at least in part on the condition of the at least one fluid(s) and/or element(s). The order of the operations, methods, mechanisms, etc. as described with respect to FIG. 6 (including FIGS. 6a, 6b, 6c, and 6d) is intended to be illustrative in nature, and not limited in scope.

One embodiment of a high-level flowchart of a defibrillation technique 2200 is described with respect to FIG. 7 and can include, but is not limited to, operations 2202 and 2204, and optional operation 2210. The high-level flowchart of FIG. 7 should be considered in combination with the embodiment of the monitoring and/or treating syringe, as described with respect to FIGS. 1-6. One embodiment of operation 2202 can include, but is not limited to, monitoring at least one fluid(s) and/or element(s) of an individual. For example, the at least one fluid(s) and/or element(s) of the individual can be monitored either within the individual, outside of the individual, or a combination thereof. One embodiment of operation 2204 can include, but is not limited to, adaptively treating the individual at least partially by determining whether to modify the at least one fluid(s) and/or element(s) within the individual at least partially in response to the monitoring the at least one fluid(s) and/or element(s) of the individual. For example, the at least one fluid(s) and/or element(s) can be modified based at least in part on the monitoring. One embodiment of operation 2210 can include, but is not limited to, wherein the adaptively treating can be performed in an absence of a physician or a medical-treating personnel. For example, the individual can adaptively treat themselves remotely from the physician or medical-treating personnel. The order of the operations, methods, mechanisms, etc. as described with respect to FIG. 7 is intended to be illustrative in nature, and not limited in scope.

In one or more various aspects, related systems include but are not limited to circuitry and/or programming for effecting the herein-referenced method aspects; the circuitry and/or programming can be virtually any combination of hardware, software, electro-mechanical system, and/or firmware configurable to effect the herein- referenced method aspects depending upon the design choices of the system designer.

4. Conclusion

This disclosure provides a number of embodiments of the monitoring and/or treating syringe mechanism. The embodiments of the monitoring and/or treating syringe mechanism as described with respect to this disclosure are intended to be illustrative in nature, and are not limiting its scope.

Those having skill in the art will recognize that the state of the art has progressed to the point where there is little distinction left between hardware, firmware, and/or software implementations of aspects of systems, such as may be utilized in the monitoring and/or treating syringe mechanism. The use of hardware, firmware, and/or software can therefore generally represent (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle can vary with the context in which the processes and/or systems and/or other technologies are deployed. For example, if an implementer determines that speed and accuracy are paramount, the implementer and/or designer of the monitoring and/or treating syringe mechanism may opt for mainly a hardware and/or firmware vehicle. In alternate embodiments, if flexibility is paramount, the implementer and/or designer may opt for mainly a software implementation. In yet other embodiments, the implementer and/or designer may opt for some combination of hardware, software, and/or firmware. Hence, there are several possible techniques by which the processes and/or devices and/or other technologies described herein may be effected, none of which is inherently superior to the other in that any vehicle to be utilized is a choice dependent upon the context in which the vehicle can be deployed and the specific concerns (e.g., speed, flexibility, or predictability) of the implementer, any of which may vary.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, and computer memory; and transmission type media such as digital and analog communication links using TDM or IP based communication links (e.g., packet links).

All of the above U.S. patents, U.S. patent application publications, U.S. patent applications, foreign patents, foreign patent applications and non-patent publications referred to in this specification and/or listed in any Application Data Sheet, are incorporated herein by reference, in their entireties.

The herein described disclosure can envision combinations of different elements that can be combined with the at least one fluid(s) and/or element(s), and thereupon injected into the individuals. Additionally, combinations of the at least one fluid(s) and/or element(s) can be withdrawn, aspirated, and/or injected. For example, certain blood transfusion patients may have their own blood removed, monitored, and depending on the condition or analysis of the blood, additional blood may be combined with the analyzed blood to at least partially form the at least one modified fluid(s) and/or element(s) that can be injected or transfused back into the individual. Such depicted architectures of the monitoring and/or treating syringe mechanism are merely exemplary, and that in fact many other architectures can be implemented which achieve the same functionality. In a conceptual sense, certain mixtures or combinations of the at least one fluid(s) and/or element(s) to achieve the same functionality can be effectively “associated” such that the desired functionality is achieved. Hence, any combination of fluid(s) and/or element(s) to achieve a particular functionality can be seen as “associated with” each other such that the desired functionality is achieved, irrespective of mixtures or intermedial fluids.

It is to be understood by those skilled in the art that, in general, that the terms used in the disclosure, including the drawings and the appended claims (and especially as used in the bodies of the appended claims), are generally intended as “open” terms. For example, the term “including” should be interpreted as “including but not limited to”; the term “having” should be interpreted as “having at least”; and the term “includes” should be interpreted as “includes, but is not limited to”; etc. In this disclosure and the appended claims, the terms “a”, “the”, and “at least one” positioned prior to one or more goods, items, and/or services are intended to apply inclusively to either one or a plurality of those goods, items, and/or services.

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that could have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that could have A alone, B alone, C alone, A and B together, A and C together, B and C together, and/or A, B, and C together, etc.).

Those skilled in the art will appreciate that the herein-described specific exemplary processes and/or devices and/or technologies are representative of more general processes and/or devices and/or technologies taught elsewhere herein, such as in the claims filed herewith and/or elsewhere in the present application.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method comprising:
analyzing at least one condition of at least one fluid as contained within an individual;
transferring at least one element relative to the individual in an effort to maintain and/or change the at least one condition of the at least one element with respect to the at least one fluid at least partially in response to the analyzing the at least one condition of the at least one fluid as contained within the individual.

2. The method of claim 1, wherein the analyzing at least one condition of at least one fluid as contained within an individual comprises:
analyzing the at least one condition of the at least one fluid as contained within the individual during anesthesia of the individual.

3. The method of claim 1, wherein the analyzing at least one condition of at least one fluid as contained within an individual comprises:
analyzing the at least one condition of the at least one fluid that can be performed or rely on information acquired externally or indirectly.

4. The method of claim 1, wherein the analyzing at least one condition of at least one fluid as contained within an individual and the transferring at least one element relative to the individual in an effort to maintain and/or change the at least one condition of the at least one fluid can be performed at least partially using an injecting-probe-based device.

5. The method of claim 1, wherein the analyzing at least one condition of at least one fluid as contained within an individual and the transferring at least one element relative to the individual in an effort to maintain and/or change the at least one condition of the at least one fluid can be performed at least partially using an injecting-probe-based device that is applied to the individual using a single injecting-probe aperture in the individual.

6. The method of claim 1, wherein the analyzing at least one condition of at least one fluid as contained within an individual and the transferring at least one element relative to the individual in an effort to maintain and/or change the at least one condition of the at least one element with respect to the at least one fluid is performed at least partially using a tubular-based device.

7. The method of claim 1, wherein the transferring at least one element relative to the individual in an effort to maintain and/or change the at least one condition of the at least one element with respect to the at least one fluid at least partially in response to the analyzing the at least one condition of the at least one fluid as contained within the individual comprises:
transferring the at least one element relative to the individual at least partially via an injecting-probe in the effort to maintain and/or change the at least one condition of the at least one element with respect to the at least one fluid.

8. The method of claim 1, wherein the transferring at least one element relative to the individual in an effort to maintain and/or change the at least one condition of the at least one element with respect to the at least one fluid at least partially in response to the analyzing the at least one condition of the at least one fluid as contained within the individual comprises:
transferring the at least one element relative to the individual at least partially via a tube in the effort to maintain and/or change the at least one condition of the at least one element with respect to the at least one fluid.

9. The method of claim 1, wherein the transferring at least one element relative to the individual in an effort to maintain and/or change the at least one condition of the at least one element with respect to the at least one fluid at least partially in response to the analyzing the at least one condition of the at least one fluid as contained within the individual comprises:
transferring the at least one element relative to the individual at least partially via a patch in the effort to maintain and/or change the at least one condition of the at least one element with respect to the at least one fluid.

10. The method of claim 1, wherein the transferring at least one element relative to the individual in an effort to maintain and/or change the at least one condition of the at least one element with respect to the at least one fluid at least partially in response to the analyzing the at least one condition of the at least one fluid as contained within the individual comprises:
filtering the at least one element relative to the individual at least partially via a patch in the effort to maintain and/or change the at least one condition of the at least one element with respect to the at least one fluid.

11. The method of claim 1, wherein the transferring at least one element relative to the individual in an effort to maintain and/or change the at least one condition of the at least one element with respect to the at least one fluid at least partially in response to the analyzing the at least one condition of the at least one fluid as contained within the individual comprises:
heating the at least one element relative to the individual at least partially via a patch in the effort to maintain and/or change the at least one condition of the at least one element with respect to the at least one fluid.

12. The method of claim 1, wherein the transferring at least one element relative to the individual in an effort to maintain and/or change the at least one condition of the at least one element with respect to the at least one fluid at least partially in response to the analyzing the at least one condition of the at least one fluid as contained within the individual comprises:
transferring at least one organism relative to the individual in the effort to maintain and/or change the at least one condition of the at least one element with respect to the at least one fluid.

13. The method of claim 1, wherein the transferring at least one element relative to the individual in an effort to maintain and/or change the at least one condition of the at least one element with respect to the at least one fluid at least partially in response to the analyzing the at least one condition of the at least one fluid as contained within the individual comprises:
transferring at least one chemical relative to the individual in the effort to maintain and/or change the at least one condition of the at least one element with respect to the at least one fluid.

14. The method of claim 1, wherein the transferring at least one element relative to the individual in an effort to maintain and/or change the at least one condition of the at least one element with respect to the at least one fluid at least partially in response to the analyzing the at least one condition of the at least one fluid as contained within the individual comprises:
transferring at least one medication relative to the individual in the effort to maintain and/or change the at least one condition of the at least one element with respect to the at least one fluid.

15. The method of claim 1, wherein the transferring at least one element relative to the individual in an effort to maintain and/or change the at least one condition of the at least one element with respect to the at least one fluid at least partially in response to the analyzing the at least one condition of the at least one fluid as contained within the individual comprises:
transferring at least one organ-directed agent relative to the individual in the effort to maintain and/or change the at least one condition of the at least one element with respect to the at least one fluid.

16. The method of claim 1, wherein the transferring at least one element relative to the individual in an effort to maintain and/or change the at least one condition of the at least one element with respect to the at least one fluid at least partially in response to the analyzing the at least one condition of the at least one fluid as contained within the individual comprises:
transferring at least one disease-directed agent relative to the individual in the effort to maintain and/or change the at least one condition of the at least one element with respect to the at least one fluid.

17. The method of claim 1, wherein the transferring at least one element relative to the individual in an effort to maintain and/or change the at least one condition of the at least one element with respect to the at least one fluid at least partially in response to the analyzing the at least one condition of the at least one fluid as contained within the individual comprises:
injecting the at least one element into the individual.

18. The method of claim 1, wherein the transferring at least one element relative to the individual in an effort to maintain and/or change the at least one condition of the at least one element with respect to the at least one fluid at least partially in response to the analyzing the at least one condition of the at least one fluid as contained within the individual comprises:
withdrawing the at least one element from the individual.

19. The method of claim 1, wherein the at least one fluid includes at least one from a group including a liquid or mixture thereof, a gas or mixture thereof, mixtures that can include solid particles in a fluid carrier, solutions, slurries, suspensions, blood, lymph, serum, urine, semen, digestive fluids, tears, ambiotic fluid, amniotic fluid, saliva, mucous, cerebro-spinal fluid, intestinal contents, bile, epithelial exudate, esophageal contents, synthetic or introduced liquids, blood substitutes or drug, nutrient, or buffered saline solutions, liquids containing dissolved gases or gas bubbles, gases containing fine liquid droplets or solid particles, gases or gaseous mixtures found within the individual that may include inhaled and exhaled air.

20. The method of claim 1, further comprising
establishing a feedback by which the at least one element can at least be partially controlled based at least in part on the at least one condition of the at least one fluid.

21. A method, comprising:
monitoring at least one fluid(s) and/or element(s) of an individual; and
adaptively treating the individual at least partially by determining whether to modify the at least one fluid(s) and/or element(s) within the individual at least partially in response to the monitoring the at least one fluid(s) and/or element(s) of the individual.

22. The method of claim 21, wherein the adaptively treating can be performed in an absence of a physician or a medical-treating personnel.

23. An apparatus, comprising:
a monitoring and/or treating syringe mechanism configurable to analyze a condition of at least one fluid; and
the monitoring and/or treating syringe mechanism configurable to determine whether to transfer an at least one modified element relative to an individual based at least in part on the monitoring and/or treating syringe mechanism configurable to analyze the condition of the at least one fluid.

24. The apparatus of claim 23, wherein the monitoring and/or treating syringe mechanism configurable to analyze a condition of at least one fluid comprises:
the monitoring and/or treating syringe mechanism configurable to analyze the condition of the at least one fluid as withdrawn from the individual.

25. The apparatus of claim 23, wherein the monitoring and/or treating syringe mechanism configurable to analyze a condition of at least one fluid comprises:
the monitoring and/or treating syringe mechanism configurable to analyze the condition of the at least one fluid as maintained and/or changed within the individual.

26. The apparatus of claim 23, wherein the monitoring and/or treating syringe mechanism configurable to analyze a condition of at least one fluid comprises:
the monitoring and/or treating syringe mechanism configurable to analyze the condition of the at least one fluid within the individual to derive analyzed information that can be at least partially transferred outside of the individual.

27. The apparatus of claim 23, wherein the monitoring and/or treating syringe mechanism configurable to determine whether to transfer an at least one modified element relative to an individual based at least in part on the monitoring and/or treating syringe mechanism configurable to analyze the condition of the at least one fluid comprises:
the monitoring and/or treating syringe mechanism configurable to determine whether to inject the at least one modified element into the individual.

28. The apparatus of claim 23, wherein the monitoring and/or treating syringe mechanism configurable to determine whether to transfer an at least one modified element relative to an individual based at least in part on the monitoring and/or treating syringe mechanism configurable to analyze the condition of the at least one fluid comprises:
the monitoring and/or treating syringe mechanism configurable to determine whether to withdraw the at least one modified element from the individual.

29. The apparatus of claim 23, wherein the monitoring and/or treating syringe mechanism configurable to determine whether to transfer an at least one modified element relative to an individual based at least in part on the monitoring and/or treating syringe mechanism configurable to analyze the condition of the at least one fluid comprises:
the monitoring and/or treating syringe mechanism configurable to determine not to transfer any fluid.

30. The apparatus of claim 23, wherein the monitoring and/or treating syringe mechanism configurable to determine whether to transfer an at least one modified element relative to an individual based at least in part on the monitoring and/or treating syringe mechanism configurable to analyze the condition of the at least one fluid comprises:
the monitoring and/or treating syringe mechanism configurable to determine whether to transfer the at least one modified element in combination with the at least one fluid into the individual.

31. The apparatus of claim 23, wherein the monitoring and/or treating syringe mechanism configurable to determine whether to transfer an at least one modified element relative to an individual based at least in part on the monitoring and/or treating syringe mechanism configurable to analyze the condition of the at least one fluid comprises:
the monitoring and/or treating syringe mechanism configurable to act as a feedback mechanism at least partially in response to the analyzing the condition.

32. The apparatus of claim 23, wherein the at least one fluid includes at least one from a group including a liquid or mixture thereof, a gas or mixture thereof, mixtures that can include solid particles in a fluid carrier, solutions, slurries, suspensions, blood, lymph, serum, urine, semen, digestive fluids, tears, ambiotic fluid, amniotic fluid, saliva, mucous, cerebro-spinal fluid, intestinal contents, bile, epithelial exudate, esophageal contents, synthetic or introduced liquids, blood substitutes or drug, nutrient, or buffered saline solutions, liquids containing dissolved gases or gas bubbles, gases containing fine liquid droplets or solid particles, gases or gaseous mixtures found within the individual that may include inhaled and exhaled air.

33. The apparatus of claim 23, wherein the at least one modified element includes at least one organism.

34. The apparatus of claim 23, wherein the at least one modified element includes heat.

35. The apparatus of claim 23, wherein the at least one modified element includes at least one chemical.

36. The apparatus of claim 23, wherein the at least one modified element includes at least one electromagnetic radiation.

37. The apparatus of claim 23, wherein the monitoring and/or treating syringe mechanism can be operated in an absence of a physician or a medical-treating personnel.

38. An apparatus, comprising:
a monitoring and/or treating syringe mechanism configurable to monitor at least one condition of at least one fluid of an individual; and
the monitoring and/or treating syringe mechanism configurable to adaptively treat the individual at least partially by determining whether to modify at least one element within the individual at least partially in response to the monitoring and/or treating syringe mechanism configurable to monitor the at least one condition of the at least one fluid of the individual.

39. The apparatus of claim 38, wherein the adaptively treating the individual at least partially includes transferring the at least one element to the individual.

40. The apparatus of claim 38, wherein the adaptively treating the individual at least partially includes withdrawing the at least one element from the individual.

41. The apparatus of claim 38, wherein the monitoring and/or treating syringe mechanism can be operated in an absence of a physician or a medical-treating personnel.
